(19) United States
(12) Patent Application Publication
(10) Pub. No.: US 2023/0390527 A1
(43) Pub. Date: Dec. 7, 2023

(54) MEDICAL DEVICES HAVING CONTROLLABLY ACTUATED ANCHORING, FRICTION REDUCTION, AND DEVICE MOVEMENT

(71) Applicants: STRYKER CORPORATION, Kalamazoo, MI (US); STRYKER EUROPEAN OPERATIONS LIMITED, Carrigtwohill, Co. Cork (IE)

(72) Inventor: Stephen Porter, Piedmont, CA (US)

(73) Assignees: STRYKER CORPORATION, Kalamazoo, MI (US); STRYKER EUROPEAN OPERATIONS LIMITED, Carrigtwohill, Co. Cork (IE)

(21) Appl. No.: 18/451,778

(22) Filed: Aug. 17, 2023

Related U.S. Application Data
(63) Continuation of application No. PCT/US2022/015864, filed on Feb. 9, 2022.
(60) Provisional application No. 63/152,330, filed on Feb. 22, 2021.

(51) Int. Cl. A61M 25/01

(52) U.S. Cl. CPC A61M 25/0158; A61M 25/0147; A61M 25/0155; A61M 2205/0283

(57) ABSTRACT

Catheter systems and method utilizing catheters having strategically configured actuators disposed thereon to modify the shape and/or stiffness of the catheter to provide electronically actuated anchoring, friction reduction, and/or motive force to move the catheter within a body lumen, such as a vascular vessel. The catheters include an elongated, flexible tubular member having one or more actuators disposed on the tubular member. The actuators are configured to modify the shape and/or stiffness of the tubular member to provide various controlled functions, including anchoring, friction reduction while moving the catheter, and/or propelling the catheter in a body lumen. The actuators may be electroactive polymer actuators which are electronically actuatable, mechanical actuators which are actuated by mechanical means such as pull wires, hydraulic or pneumatic actuators which are actuatable by fluid pressure, or combinations thereof.

### RELATED APPLICATION DATA

This application is a continuation of International Patent Application No. PCT/US2022/015864, filed on Feb. 9, 2022, pending, which claims priority to and the benefit of U.S. Provisional Patent Application No. 63/152,330, filed on Feb. 22, 2021, lapsed. The entire disclosures of the above applications are expressly incorporated by reference.

### FIELD OF THE INVENTION

The disclosed inventions generally relate to medical devices and methods for performing procedures within a lumen of a vascular system of a patient, and more particularly, to catheters, sheaths and or guidewires having actuators, such as mechanical, hydraulic, thermo resistive or electroactive polymer actuators, to modify the stiffness and/or shape of a catheter to provide actuatable functionality, such as a force induced or electronically actuatable functionality, for use within a vascular system, and methods of using the same.

### BACKGROUND

Various designs of medical devices have been previously disclosed for performing a variety of medical procedures, including interventional therapy, drug delivery, diagnosis, perfusion, and the like. In general, medical sheaths, catheters or guidewires are used by introducing a distal end of the device through an entry site of a patient and into the vascular system of the patient, such as a vein or artery. For example, a catheter is advanced from the entry site by guiding and pushing a proximal portion of the catheter to advance the catheter through the vascular system to a target site for performing a therapeutic and/or diagnostic medical procedure. The vascular path to the target site is often tortuous, requiring the catheter to follow a curving and direction changing path. The user (typically a physician) must steer the catheter as it is advanced by manipulating the catheter (e.g., twisting, rotating, etc.) and/or by using pull wires or other steering mechanism coupled to the distal end to steer the distal end along the vascular path.

An example of one type of intravascular catheter is a guide catheter. A guide catheter is used to guide other instruments to a desired site within a vascular system. Generally, a guide catheter includes an elongated, flexible, tubular member, and may have a steering mechanism such as pull wires. One common type of guide catheter is a balloon guide catheter which also includes a balloon member affixed to the tubular member. The balloon may be affixed to a distal portion of the tubular member or other suitable location. The balloon can provide any of several useful functions, including anchoring the guide catheter in place once it is advanced to the target site, sealing the vascular lumen at the location of the balloon (e.g., to block fluid flow), and/or performing a therapeutic procedure such as enlarging the vessel, compressing an occlusion, etc. A balloon guide catheter also includes an inflation lumen extending from a proximal end of the tubular member to the inflatable balloon for injecting inflation fluid to inflate the balloon and also to release the fluid pressure to deflate the balloon.

Various designs for steering and changing the shape of a catheter have been previously disclosed. Some of these designs utilize electroactive polymer actuators disposed on the catheter and a control unit coupled to the actuators to selectively send electrical control signals to the actuators which cause the actuators to change the configuration of the catheter. For example, U.S. Pat. No. 7,261,686 describes a guide catheter having electroactive polymer actuators disposed along its axial length in which, based upon the control signals received from the control unit, the actuators change the shape and/or stiffness of the guide catheter. U.S. Pat. No. 7,261,686 explains that the actuators can be used to shape of the guide catheter to fit the patient anatomy, to controllably steer the catheter as it is inserted, and to stiffen the catheter. Similarly, U.S. Pat. No. 8,920,870, describes a catheter system including a catheter having sections of electroactive polymer at different locations on the catheter which are actuated by a control signal to hinge, bend, rotate, expand or contract the sections of the catheter, or to cause a region surrounding a section to increase in stiffness, thereby increasing the pushability of the catheter.

### SUMMARY

The disclosed inventions are directed to devices having strategically configured and positioned actuators disposed thereon to provide actuated anchoring, friction reduction and/or motive force to move the device within a body lumen, such as a vascular vessel. The actuators may be electromechanical actuators actuated by electrical signals, mechanical actuators which are actuated by mechanical means such as pull wires, hydraulic or pneumatic actuators which are actuatable by fluid pressure, or combinations thereof. While the description of the disclosed embodiments are directed to guide catheters used for insertion and positioning of therapeutic devices within a vascular system of a patient, it is understood that the disclosed inventions are not limited to guide catheters, but may be used with any suitable catheter, sheath or guidewire. For instance, the catheters described herein may be any suitable type of device, including guide catheters, balloon guide catheters, diagnostic catheters, therapeutic catheters, access sheaths, guide sheaths, dilators, guidewires, stylets, etc.

Accordingly, in one embodiment, a catheter system comprises an elongated, flexible tubular member, having an anchoring portion with an intrinsic flexibility, and one or more actuators disposed on the tubular member. As used herein, the term “disposed on” means that a first structure is attached on, integrated with, applied to, or otherwise affixed to, a second structure. The tubular member has an anchoring portion having an intrinsic flexibility. For example, the tubular member is typically very flexible in order to navigate a tortuous path within a body lumen. The one or more actuators may be disposed along the axial length, as well as at different perimeter/circumferential positions, of the tubular member. The one or more actuators are configured to modify a stiffness of the anchoring portion into an anchoring shape configured to anchor the anchoring portion in place in a body lumen corresponding to the anchoring shape, when the actuators are actuated. The one or more actuators have a non-actuated state in which the one or more actuators allow the tubular member to have its intrinsic flexibility, and an actuated state when the actuators are actuated in which the one or more actuators stiffen the anchoring portion into an anchoring shape configured to anchor the anchoring portion in place in a body lumen corresponding to the anchoring shape.

The catheter system has a non-actuated configuration wherein the one or more actuators are non-actuated such that the actuators do not significantly affect the stiffness and/or shape of the anchoring portion of the tubular member. In other words, the anchoring portion of the tubular member has its relaxed shape and/or intrinsic flexibility. The catheter system also has an actuated configuration wherein the one or more actuators are actuated which causes the actuators to stiffen the anchoring portion in the anchoring shape configured to anchor the anchoring portion in place in a body lumen corresponding to the anchoring shape. The anchoring shape is configured to anchor the anchoring portion in place in a body lumen corresponding to the anchoring shape. The anchoring resists movement of the tubular member thereby preventing the tubular member from moving longitudinally within the body lumen. Accordingly, instruments and other catheters can be navigated and manipulated through the tubular member while the tubular member is anchored in place by the anchoring shape.

For example, the body lumen may have a curved shape (e.g., an S-shape to correspond to the petrous carotid artery or a path traversing the aortic arch and into a branch vessel) such that when the anchoring portion is stiffened in the curved shape of the body lumen, the anchoring portion anchors the tubular member in place such that it resists being moved longitudinally.

In various embodiments of the catheter system, the one or more actuators may be configured to also modify the shape of the anchoring portion. For instance, the actuators may be configured to also form the anchoring portion into the anchoring shape. Hence, the actuators both form the anchoring portion into the anchoring shape, and stiffen the anchoring portion in the anchoring shape. In the example above for an S-shape, the actuators are configured to form the anchoring portion of the tubular member into an anatomically corresponding S-shape.

In various embodiments of the catheter system, the one or more actuators may comprise mechanical actuators which are actuators by one or more pull wires coupled to the mechanical actuators. In one embodiment, each mechanical actuator comprises an actuator element having an anisotropic region about a center axis of the tubular member with a first area having a first compliance and a second area having a second compliance different from the first compliance such that a compressive loading on the actuator element by the one or more pull wires causes the actuator element to bend and stiffen into a predetermined shape. The anisotropic regions may be formed by a tube, such as a hypotube, having cut patterns which confer a first compliance to the first area and a second compliance to the second area.

In other embodiments of the catheter system, the actuators may have other types of mechanical actuators actuated by mechanical means such as pull wires, hydraulic or pneumatic actuators which are actuatable by fluid pressure, or combinations thereof. Some representative examples include the following. Actuator elements which are mechanically driven using linear tensile or compressive elements incorporated within the device, such that when a force is applied to the elements, the force induces an asymmetrical elastic length contraction or expansion of discrete structures within the device at the location of the desired shape change. In another example, the actuators are mechanically driven using rotatable concentric, precurved, elastic tubes which constitute the primary reinforcement of the device, wherein upon rotation and/or extension of the tubes with respect to each other their inherent curvatures reinforce or cancel elastically to change the shape of the device along its length.

In one example of hydraulic actuators, the actuators are hydraulically driven via fluid connection embedded in the device to allow inflation or evacuation of fluid cavities incorporated within the device to impart a dimensional change via one or more of: direct pressure induced elastic length expansion or contraction of asymmetrically placed elements and/or cavities within the device at the location of the desired shape change; and/or osmotically pressure driven elastic length expansion or contraction of asymmetrically placed cavities within the device and separated from the fluid cavities by a membrane at the location of the desired shape change.

In additional embodiments of the catheter system, the actuators may be other types of electrical actuators. For example, the actuators may be electrically driven using conductors embedded in the device to induce a bending force via one or more of: application of a potential difference across a resistive material to change the temperature and thereby change the shape of a shape memory material; application of a potential difference across an electroactive polymer and thereby change the shape of the electroactive polymer; and/or application of a potential difference across a piezoelectric material and thereby change the shape of the piezoelectric material.

Another disclosed embodiment is directed to a method of using the catheter system. The method includes inserting a distal end of the catheter system and advancing the catheter into the body lumen to a target location with the one or more actuators non-actuated and the anchoring portion having its intrinsic flexibility. The actuators are actuated thereby stiffening the anchoring portion in the anchoring shape and anchoring the anchoring portion in place in the body lumen. The stiffened anchoring portion anchors the catheter in place within the body lumen. This maintains the catheter in position so that other catheters and/or instruments can be inserted through the catheter without the catheter moving from its anchored position.

The method may also include the actuation of the actuators forming the anchoring portion into the anchoring shape, in the case where the actuators are configured to also form the anchoring portion into the anchoring shape.

In still additional disclosed embodiments, the actuators comprise electroactive polymers. The use of electroactive polymers which are electronically actuatable allows the catheter to be electronically controlled, which may also include computerized control. Localized electronic actuation of the electroactive polymer actuators also has minimal effect on remote portions of the catheter such as the proximal shaft, as opposed to pull wire technology. The use of electroactive polymer actuators also enables the use of many actuators, and greater complexity of placement of the actuators. The use of electronic actuation also enables the catheter to have programmable dynamic shapes and configurations, and the additional ability to sense the strain environment at the actuator by measuring a capacitance or induced voltage of the electroactive polymer when in an non activated state.

In these embodiments, the catheter systems utilize strategically configured electroactive polymer actuators disposed on and/or within a catheter to provide electronically actuated anchoring, friction reduction, and motive force to move the catheter within a body lumen, such as a vascular vessel. The use of electroactive polymers which are electronically actuatable allows the catheter to be electronically controlled, which may also include computerized control. Localized electronic actuation of the electroactive polymer actuators also has minimal effect on remote portions of the catheter such as the proximal shaft, as opposed to pull wire technology. The use of electroactive polymer actuators also enables the use of many actuators, and greater complexity of placement of the actuators. The use of electronic actuation also enables the catheter to have programmable dynamic shapes and configurations. The use of electronic actuation also enables the catheter to have strain sensing capability and feedback control to induce catheter shapes that match or mimic the surrounding anatomical lumen shape.

The electroactive polymer actuators employed in the embodiments described herein utilize electroactive polymers, sometimes referred to as “conducting polymers.” Actuators utilizing electroactive polymers are advantageous in the catheters disclosed herein because they can be very small, yet produce large forces and strains. They are also relatively low cost, and relatively easy to incorporate into the catheters disclosed herein.

Electroactive polymers are a form of polymers having the ability to change shape in response to electrical stimulation, i.e., an electrical signal. Dimensional changes in the electroactive polymer may be produced by the mass transfer of ions into or out of the polymer. Hence, the transfer of ions into or out of the electroactive polymer results in a dimensional change, such as expansion or contraction due to ion insertion between polymer chains, and/or inter-chain repulsion. Thus, electroactive polymer actuators are produced using electroactive polymers based on these characteristics. The electroactive polymer actuators are actuated and de-actuated by applying and removing an electrical control signal to the actuator. The actuators may then be applied to a structure to exert forces on the structure to exert force on the structure which may change the physical properties of the structure, such as changing the shape, dimension, stiffness, and/or position of the structure.

In an exemplary embodiment utilizing electroactive polymer actuators, a catheter system includes a catheter having an elongated, flexible tubular member and one or more electroactive polymer actuators disposed on the tubular member. The tubular member has an anchoring portion having an intrinsic flexibility. For example, the tubular member is typically very flexible in order to navigate a tortuous path within a body lumen. The one or more electroactive polymer actuators may be disposed along the axial length, as well as at different perimeter/circumferential positions, of the tubular member. The one or more electroactive polymer actuators are configured to modify a stiffness of the anchoring portion in an anchoring shape configured to anchor the anchoring portion in place in a body lumen corresponding to the anchoring shape, in response to one or more electrical control signals.

Accordingly, the catheter system has a non-actuated configuration in which the electroactive polymer actuators are non-actuated. In other words, the electroactive polymer actuators are not actuated by an electrical signal, such as a voltage and/or current, such that they are in a relaxed configuration which does not significantly affect the stiffness and/or shape of the anchoring portion of the tubular member, i.e., the anchoring portion of the tubular member has its intrinsic flexibility.

The catheter system also has an actuated configuration wherein the one or more electroactive polymer actuators are actuated by one or more control signals, such as a voltage and/or current. The control signal causes the electroactive polymer actuators to stiffen the anchoring portion in the anchoring shape, i.e., the actuators increase the stiffness of the anchoring portion from the intrinsic flexibility of the tubular member. The anchoring shape is configured to anchor the anchoring portion in place in a body lumen corresponding to the anchoring shape. This allows instruments and other catheters to be navigated and/or manipulated through the tubular member with the tubular member kept stationary by the anchoring shape.

As one example, the body lumen may have a curved shape (e.g., an S-shape to correspond to the petrous carotid artery) such that when the anchoring portion is stiffened in the curved shape of the body lumen, the anchoring portion anchors the tubular member in place such that it resists being moved longitudinally. Other catheters and instruments can then be guided through the tubular member to a target site within the body lumen while the catheter remains anchored in place.

In various embodiments of the catheter system, the one or more electroactive polymer actuators may be configured to also modify the shape of the anchoring portion. For example, the electroactive polymer actuators may be configured to also form the anchoring portion into the anchoring shape. In this way, the electroactive polymer actuators both form the anchoring portion into the anchoring shape, and stiffen the anchoring portion in the anchoring shape. In the example above for an S-shape, the electroactive polymer actuators are configured to form the anchoring portion of the tubular member into an S-shape.

In various other embodiments, the catheter system may further comprise a controller operably coupled to the plurality of electroactive polymer actuators. The controller is configured to selectively transmit the electrical control signals to the plurality of electroactive polymer actuators for modifying the configuration of the tubular member, such as modifying the stiffness and/or shape of the anchoring portion. The electrical control signals may be a voltage applied to each of the electroactive polymer actuators. The controller may be coupled to the one or more electroactive polymer actuators using a plurality of conductors, such as wires, conductive traces, or the like, disposed on the tubular member. Each conductor has a first end connected to a respective electroactive polymer actuator and a second end connected to the controller.

In another embodiment the controller may be able to sense the strain applied to the actuators via an induced voltage or a change in capacitance and thereby interpret the device curvature in the location of the sensed actuators and thereby interpret the anatomical shape in which the unactuated device resides. The controller can then apply signals to the sensed actuators to induce a shape change which mimics the interpreted anatomical lumen shape, thereby achieving an anchoring shape which is adapted to the anatomical shape without a priori knowledge of the anatomical shape of the anchoring location.

In various embodiments, the electroactive polymer actuators may comprise a parylene film. In other embodiments, the electroactive polymer actuators comprise any suitable electroactive polymer, such as parylene, polyaniline, polypyrrole, polysulfone, or polyacetylene.

Another aspect of the disclosed invention is directed to a method of using the above-described exemplary embodiments of a catheter system. The method includes inserting a distal end of the catheter into a body lumen and advancing the catheter to a target location within the body lumen with the one or more electroactive polymer actuators non-actuated such that the anchoring portion of the tubular member has its intrinsic flexibility. This allows the catheter to be advanced through the body lumen in a flexible state. Once the catheter is positioned at the target location, the electroactive polymer actuators are actuated using a control signal which stiffens the anchoring portion in the anchoring shape. The stiffened anchoring portion anchors the catheter place in the body lumen. This maintains the catheter in position so that other catheters and/or instruments can be inserted through the catheter without the catheter moving from its anchored position.

The method may also include the actuation of the electroactive polymer actuators forming the anchoring portion into the anchoring shape, in the case where the electroactive polymer actuators are configured to also form the anchoring portion into the anchoring shape.

The method may further include any one or more of the other aspects of the above-described catheter system.

Another embodiment of a catheter system utilizing electroactive polymer actuators is directed to a catheter system configured to utilize electroactive polymer actuators to reduce static friction of the catheter as it is moved longitudinally within a body lumen. In this embodiment, the catheter system includes a catheter having an elongated, flexible tubular member and a plurality of electroactive polymer actuators disposed on the tubular member. The plurality of electroactive polymer actuators are configured to form the tubular member into a substantially sinusoidal shape and to cycle the sinusoidal shape back and forth between a substantially sinusoidal shape 0° out of phase and a substantially sinusoidal shape 180° out of phase (i.e. transition the sinusoidal shape back and forth from 0° to 180° out of phase). In other words, the shape of the tubular member cycles such that peaks of the sinusoidal shape move to be valleys, and vice versa.

The catheter system also includes a controller operably coupled to the plurality of electroactive polymer actuators. The controller is configured to transmit control signals to the plurality of electroactive polymer actuators to dynamically cycle the tubular member back and forth between the substantially sinusoidal shape 0° out of phase and the substantially sinusoidal shape 180° out of phase.

Accordingly, when the catheter system is moved longitudinally within a body lumen, the plurality of electroactive polymer actuators are actuated to dynamically cycle the tubular member back and forth between a substantially sinusoidal shape 0° out of phase and a substantially sinusoidal shape 180° out of phase. This cycling sinusoidal movement of the tubular member breaks the static friction of the tubular member as the catheter is moved longitudinally thereby requiring less force to move the catheter and making the movement of the catheter smoother.

In various embodiments of the friction reduction embodiment, the controller is configured to dynamically cycle the sinusoidal shape of the tubular member at an amplitude, a peak to peak wavelength and cycling frequency which reduces static friction of the tubular member as it is moved longitudinally within a body lumen. For instance, the amplitude and wavelength may be set so that it is just sufficient to break static friction, while the frequency is such that it does not damage the walls of the body lumen or create other unwanted side effects.

In various embodiments of the friction reduction embodiment, the electroactive polymer actuators are arranged in one specific configuration which provides the sinusoidal shape. The plurality of electroactive polymer actuators comprises a first set of electroactive polymer actuators and a second set of electroactive polymer actuators. On a first side of the tubular member, the electroactive polymer actuators of the first set are disposed longitudinally along the tubular member and alternating with electroactive polymer actuators of the second set disposed longitudinally along the tubular member on the first side. Similarly, on a second side of the tubular member opposite the first side, the electroactive polymer actuators of the first set are disposed longitudinally along the tubular member and alternating with electroactive polymer actuators of the second set disposed along the tubular member on the second side. In addition, the electroactive polymer actuators of the first set are arranged such that the electroactive polymer actuators on the first side are offset from the electroactive polymer actuators on the second side. Similarly, the electroactive polymer actuators of the second set are arranged such that the electroactive polymer actuators on the first side are offset from the electroactive polymer actuators on the second side. In one way, the electroactive polymer actuators of the first set on the first side oppose the electroactive polymer actuators of the second set on the second side, and the electroactive polymer actuators of the first set on the second side oppose the electroactive polymer actuators of the second set on the first side, and vice versa. The configuration of the electroactive polymer actuators is such that actuation of the first set of electroactive polymer actuators modifies the shape of the tubular member into the substantially sinusoidal shape 0° out of phase, and actuation of the second set of electroactive polymer actuators modifies the shape of the tubular member into the sinusoidal shape 180 out of phase.

In another aspect of the friction reduction embodiment, the first set of electroactive polymer actuators are electrically connected to the controller via a first pair of electrical conductors such that all of the electroactive polymer actuators in the first set are actuated by a single first signal from the controller. Likewise, the second set of electroactive polymer actuators are connected to the controller via a second pair of electrical conductors such that all of the electroactive polymer actuators in the second set are actuated by a single second signal from the controller. The second pair of electrical conductors are electrically insulated/separated from the first pair of electrical conductors. Accordingly, the plurality of electroactive polymer actuators may be actuated to dynamically cycle the tubular member back and forth between the substantially sinusoidal shape 0° out of phase and the substantially sinusoidal shape 180° out of phase by alternating between the first signal and second signal.

Another aspect of the disclosed embodiments is directed to a method of using the friction reduction embodiment of a catheter system in a body lumen. The method comprises inserting the catheter into a body lumen. The catheter is moved longitudinally in the body lumen while the controller transmits control signals to the plurality of electroactive polymer actuators thereby dynamically cycling the tubular member back and forth between the substantially sinusoidal shape 0° out of phase and the substantially sinusoidal shape 180° out of phase. The cycling sinusoidal motion of the tubular member breaks the static friction of the tubular member as the catheter is moved longitudinally.

In various embodiments, the method of using the friction reduction embodiment of a catheter further includes any one or more of the other aspects of the above-described second embodiment of a catheter system.

Another embodiment of a catheter system utilizing electroactive polymer actuators is directed to a catheter system configured to utilize an electroactive polymer actuator to shape the tubular member to make substantially continuous circumferential contact with a wall of a body lumen to stabilize the catheter. The catheter system includes an elongated, flexible tubular member and an electroactive polymer actuator disposed on the tubular member in a helical path along the tubular member. Actuation of the helically placed electroactive polymer actuators using an electrical control signal transitions a shape of the tubular member into a substantially helical shape such that the tubular member can make circumferential contact with a wall of a body lumen along a length of tubular member sufficient to stabilize the tubular member in a body lumen. For example, the circumferential contact can prevent longitudinal movement of the catheter in the body lumen, similar to the anchoring feature of the first embodiment, and/or center the catheter in the body lumen, or otherwise prevent movement of the catheter.

In various embodiments of the helical shaped embodiment, the catheter is configured such that the tubular member in the substantially helical shape makes circumferential contact with a wall of a body lumen along a selected discrete length of the catheter or along substantially an entire insertable length of the tubular member. For example, the catheter may be configured with the electroactive polymer along a length starting at a location at a first distance from the distal end of the catheter and ending at a second longer length from the distal end of the catheter or the catheter may be configured with the electroactive polymer along substantially the entire length of the tubular member, except for a proximal end which remains outside the body.

In various embodiments of the helical shaped embodiment, the catheter system may further comprise a controller operably coupled to the electroactive polymer actuator, the controller configured to selectively transmit the control signal to the electroactive polymer actuator.

In various embodiments of a helical shaped device, the one or more actuators may comprise mechanical actuators which are actuated by one or more pull wires coupled to the mechanical actuators. In one embodiment, a series of actuatable mechanical structures are disposed serially along a length of the device, each mechanical structure comprises an anisotropic actuatable element (and therefore a preferred bending direction relative to the axis) wherein the actuatable elements are rotationally indexed in series about and along a center axis of the device in a helical path such that a compressive loading on the series of actuator elements by the one or more pull wires causes the device to bend and stiffen into a helical shape. The series of mechanical structures and corresponding anisotropic elements may be formed within a tube, such as a hypotube, having cut patterns which confer an anisotropic compliance to the region of each individual structure which can be the same or different from adjacent structures thereby enabling each structure to bend in the same or different direction.

In another embodiment, a series or continuum of mechanical structures are disposed along a length of the device, each mechanical element or the continuum having isotropic bending about a center axis of the device (and therefore a non-preferred bending direction relative to the axis) and a single pull wire arranged in a helical path along the length of the series or continuum of mechanical structures such that a compressive loading on the series or continuum of mechanical structures by the pull wire causes the device to bend and stiffen into a helical shape. The series or continuum of mechanical structures and corresponding isotropic elements may be formed by a coil; alternatively it may be formed within a tube, such as a hypotube, having cut patterns which confer an isotropic compliance to the region of each individual structures which enabling each structure to non-preferentially bend in any direction.

In other embodiments of the helical shaped device, the actuators may have other types of mechanical actuators actuated by mechanical means such as pull wires, hydraulic or pneumatic actuators which are actuatable by fluid pressure, or combinations thereof.

In preferred embodiments the helical shaped device, the helical shape has an outer helix diameter and a helix pitch. The outer helix diameter is selected to be at least as large as the target lumen in which anchoring is to take place, and more preferred the outer helix diameter is between 1.25 and 2 times the target lumen diameter. The helix pitch is selected to be short enough to confer an amount of friction between the wall of the lumen and the helically shaped device capable of anchoring the device in place in addition the helix pitch needs to be long enough to not impede smooth passage of other catheters and/or instruments either through the lumen of the device or unsheathing the exterior of the helical device. A preferred helix pitch is between 3 and 10 times the outer helix diameter.

Another aspect of the disclosed inventions is directed to a method of using the helical shaped embodiment of a catheter system in a body lumen. The method includes inserting the catheter into a body lumen with the electroactive polymer actuator in a non-actuated configuration with the tubular member having its inherent flexibility and relaxed shape. The electroactive polymer actuator is actuated using a control signal thereby transitioning the shape of the tubular member into a substantially helical shape such that the tubular member makes circumferential contact with a wall of a body lumen along a length of tubular member sufficient to stabilize the tubular member in a body lumen. Accordingly, the catheter is stabilized within the body lumen. In various embodiments of the method, the tubular member may make circumferential contact with a wall of a body lumen along substantially an entire insertable length of the tubular member.

Another embodiment of a helical shaped device is directed to a device which utilizes actuators to create motive force to move the catheter longitudinally within a body lumen. The catheter system includes an elongated, flexible member. The flexible member has a plurality of radially arranged linear portions or path segments. The segments are longitudinally coincident along a discrete length of the device and radially spaced apart from each other, such as a first segment, a second segment radially displaced from the first segment and a third segment radially displaced from the second segment, etc. A respective actuator or series of actuators is disposed within each respective segment in a helical path along the discrete length of the device such that actuation of the respective actuator or actuators using a respective control method transitions a shape of the discrete length of the device into a substantially helical shape. Each respective actuator or series of actuators is configured such that modulated actuation of the actuators creates a friction force between the flexible member and the wall of the body lumen. Serial activation of the actuator or series of actuators within each of the path segments moves the flexible member longitudinally within the body lumen. For instance, the modulated actuation of the path segments can cause the flexible member to move in a worm crawler-like motion or a rotating screw motion which creates a motive force to move the flexible member longitudinally.

In various embodiments, using a respective control method, a modulated signal or force is imposed upon the actuator or series of actuators within each of the path segments thereby controlling the amplitude of the shape response for each respective path segment. The control imparts sine wave amplitude modulation of signals or forces, with each signal or force being out of phase from the other signals. For instance, in the case of three path segments, each signal or applied force may be 120° out of phase from the other two signals or applied forces.

In various embodiments, devices utilizing electroactive polymer actuation may further comprise a controller operably coupled to the electroactive polymer actuators and configured to transmit the respective control signals to the respective electroactive polymer actuators. Likewise, devices utilizing mechanical or hydraulic actuation may further comprise a controller operably coupled to the mechanical or hydraulic actuators and configured to transmit the respective control forces or pressures to the respective actuators. The controller may be electro-mechanical or purely mechanical and capable of generating modulated forces and or pressures required for modulated actuation of the respective actuators.

Another aspect of the disclosed embodiments is directed to a method of using the helical shape device embodiment in a body lumen. The method includes inserting the catheter system into a body lumen. The respective actuator or series of actuators within each of the path segments are actuated using a respective modulated control method to each respective path segments. The modulated actuation creates a motive force between the flexible member and a wall of the body lumen which moves the flexible member longitudinally within the body lumen.

In various embodiments, the respective modulated control signals or forces are sine wave amplitude modulation signals or applied forces, with each signal or applied force being out of phase from the other signals or applied forces. For instance, in the case of three path segments, each signal or applied force is 120° out of phase from the other signals or applied forces.

Accordingly, embodiments described herein provide innovative devices utilizing actuators, such as mechanical actuators and/or electroactive polymer actuators and methods of using the same which provide actuated anchoring, friction reduction and/or motive force to move the device within a body lumen. The electroactive polymer actuated embodiments provide an electronically controllable device which allows for computerized control, localized electronic actuation of the electroactive polymer actuators having minimal effect on remote portions, the use of many actuators, greater complexity of placement of the actuator, and also enables programmable and/or dynamic shapes and structures of the devices.

### BRIEF DESCRIPTION OF THE DRAWINGS

The foregoing, along with other and further embodiments and aspects of the disclosed inventions, will now be described in greater detail in the below detailed description, to be read in view of the accompanying figures, wherein like reference numerals refer to like elements and the description for like elements shall be applicable for all described embodiments wherever relevant.

FIG. 1 is a schematic view of a catheter system, in accordance with embodiments of the disclosed inventions;

FIGS. 2A-2C are perspective views of a portion of various configurations of actuators on catheters for use in the catheter system of FIG. 1, in accordance with embodiments of the disclosed inventions;

FIGS. 3A-3B are partial side views of a configuration of an electroactive polymer actuator on a catheter for use in the catheter system of FIG. 1, in accordance with embodiments of the disclosed inventions;

FIGS. 4A-4B are side views illustrating an anchoring feature of the catheter for use in the catheter system of FIG. 1, in accordance with embodiments of the disclosed inventions;

FIGS. 5A-5B are side views illustrating another anchoring feature of the catheter for use in the catheter system of FIG. 1, in accordance with embodiments of the disclosed inventions;

FIG. 6 illustrates a flow chart of a method of using the catheters having anchoring features as shown in FIGS. 4A-4B and 5A-5B, in accordance with embodiments of the disclosed inventions;

FIGS. 7A-7B are side views of another catheter for use in the catheter system of FIG. 1, in accordance with embodiments of the disclosed inventions;

FIGS. 8A-8C illustrate the dynamic cycling motion of the catheter of FIGS. 7A-7B accordance with embodiments of the disclosed inventions;

FIG. 9 illustrates a flow chart of a method of using the catheter of FIGS. 7A-7B, in accordance with embodiments of the disclosed inventions;

FIG. 10A is a side view of another catheter for use with the catheter system of FIG. 1, accordance with embodiments of the disclosed inventions;

FIG. 10B is a side view of the catheter of FIG. 10A in an actuated configuration, accordance with embodiments of the disclosed inventions;

FIG. 10C is a side, perspective view of the catheter of FIG. 10A in a non-actuated configuration;

FIG. 10D is a side, perspective view of the catheter of FIG. 10A in an actuated configuration;

FIG. 11 is a side view the catheter of FIG. 10A in an actuated configuration in a body lumen, accordance with embodiments of the disclosed inventions;

FIG. 12 illustrates a flow chart of a method of using the catheter of FIGS. 10A-10D, in accordance with embodiments of the disclosed inventions;

FIG. 13 is a side view of still another catheter for use with the catheter system of FIG. 1, in accordance with embodiments of the disclosed inventions;

FIGS. 14A-14D are side views of the catheter of FIG. 13 in various states of a modulated actuation sequence for producing a motive force, in accordance with embodiments of the disclosed inventions;

FIG. 14E is a graph of a modulated actuation sequence for the catheter of FIG. 13 to produce the modulated actuation sequence depicted in FIGS. 14A-14D, in accordance with embodiments of the disclosed inventions; and

FIG. 15 is a side view of still another catheter for use with the catheter system of FIG. 1, in accordance with embodiments of the disclosed inventions;

FIGS. 16A-16C are side views of the catheter of FIG. 15 in various states of a modulated actuation sequence for producing a motive force, in accordance with embodiments of the disclosed inventions;

FIG. 16D is a graph of a modulated actuation sequence for the catheter of FIG. 15 to produce the modulated actuation sequence depicted in FIGS. 16A-16C, in accordance with embodiments of the disclosed inventions;

FIG. 17 illustrates a flow chart of a method of using the catheters of FIGS. 13 and 15, in accordance with embodiments of the disclosed inventions.

FIG. 18 is a side view of a portion of a catheter reinforcement having a plurality of mechanical actuators for use in the catheter system, in accordance with embodiments of the disclosed inventions;

FIG. 19 is an illustration of the catheter reinforcement of FIG. 16 under no compressive load which shows the areas of varying compliance provided by the mechanical actuators, in accordance with embodiments of the disclosed inventions;

FIG. 20 is an illustration of the catheter reinforcement of FIG. 16 under a compressive load which shows the bending curvature/stiffening of the areas of varying compliance provided by the mechanical actuators, in accordance with embodiments of the disclosed inventions;

FIG. 21 is an enlarged cross-section of one embodiment of the catheter reinforcement of the FIG. 18, showing one embodiment of a pull wire actuator for the mechanical actuators, in accordance with embodiments of the disclosed inventions;

FIG. 22 is an enlarged cross-section of another embodiment of the catheter reinforcement of FIG. 18, showing another embodiment of a multiple pull wire actuator for the mechanical actuators, in accordance with embodiments of the discloses inventions;

FIG. 23 is an enlarged side view of one of the mechanical actuators of the catheter reinforcement with no compressive load, in accordance with embodiments of the discloses inventions; and

FIG. 24 is an enlarged side view of the mechanical actuator of FIG. 21 which shows the bending of the actuator under a compressive load, in accordance with embodiments of the discloses inventions.

### DETAILED DESCRIPTION

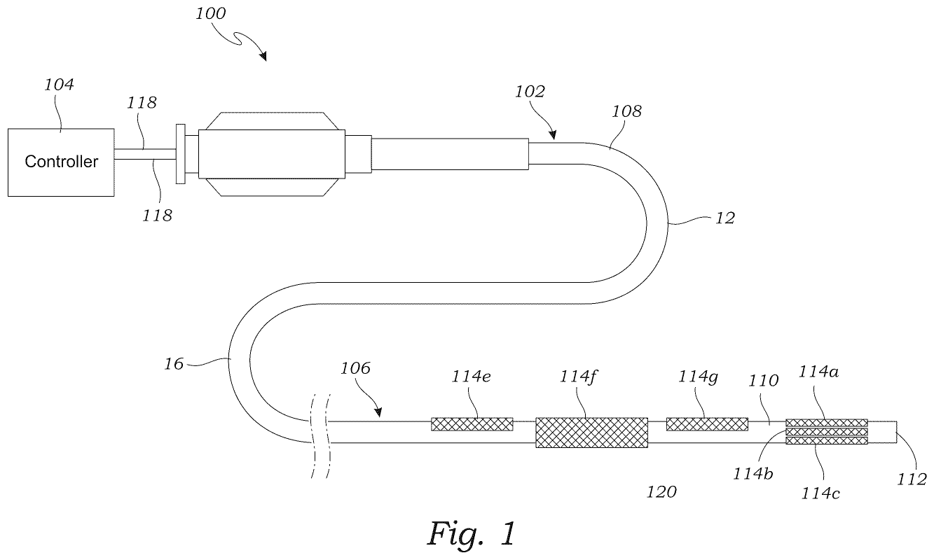

FIGS. 1-4 illustrate a catheter system 100 configured in accordance with an exemplary embodiment of the disclosed inventions. The catheter system 100 is configured generally for performing a procedure, such as a medical treatment or diagnostic procedure, within a body lumen, such as a lumen within the vascular system. The catheter system 100 includes a catheter 102 and a controller 104. The catheter 102 includes a flexible, elongated tubular member 106 having a proximal portion 108 and a distal portion 110 and a working lumen 112 extending therebetween. The tubular member 106 has an intrinsic flexibility (or an intrinsic stiffness) dependent on the physical properties and dimensions of the material(s) which form the tubular member 106. The intrinsic flexibility is typically very flexible so that the catheter 102 can navigate relatively tortuous paths of a body lumen of a vascular system. The catheter 102 also has one or more actuators 114 disposed on the tubular member 106.

The actuators 114 may include steering actuators 114a, 114b, 114c and 114d (not shown in the figures but positioned opposite the steering actuator 114b on the tubular member 106) positioned on the distal portion of the tubular member 106, and anchoring actuators 114e, 114g, 114f. More or fewer actuators 114 may be utilized, as any suitable number of actuators 114 may be disposed on the tubular member 106, depending on the desired actuated configuration of the catheter 102, as described herein. The actuators 114 may comprise electroactive polymer materials. As described herein, electroactive polymers have the ability to change shape in response to electrical stimulation, i.e., an electrical signal. The electroactive polymer may be an electroactive polymer film, or other suitable form of electroactive polymer. The electroactive polymer may be any suitable electroactive polymer, such as parylene, polyaniline, polypyrrole, polysulfone, or polyacetylene. The electroactive polymer actuators 114 are actuated by applying an electrical control signal to the actuator and de-actuated by removing the electrical control signal.

The actuators 114 are applied to the tubular member 106 to exert forces on the structure of the tubular member to exert forces on the tubular member 106 which change the physical properties of the tubular member 106, such as changing the shape, dimension, stiffness, and/or position of the tubular member 106.

The actuators 114 may be disposed on the tubular member 106 by any suitable method. As some examples, the actuators 114 may be attached onto a surface of the tubular member 106 (e.g., onto the outer surface, onto the inner surface) using an adhesive, or other fastening means. The actuators 114 may be deposited onto the tubular member 106, such as by a spray deposition, electro-deposition, or other suitable deposition means. The actuators 114 may be integrated with, or into, the tubular member 106, such as being molded within the wall of the tubular member 106.

The catheter 102 has a non-actuated configuration in which the actuators 114 are not actuated by a control signal. In the non-actuated configuration, each of the actuators 114 and the respective segment of the tubular member 106 on which the respective actuator 114 is disposed are in a relaxed configuration (i.e., non-actuated configuration). In the relaxed configuration the actuators 114 do not significantly affect the configuration (e.g., the stiffness and/or shape) of the tubular member 106. Accordingly, in the relaxed configuration, the tubular member 106 has its intrinsic shape and flexibility. The catheter 102 has an actuated configuration wherein one or more of the electroactive polymer actuators are actuated by one or more electrical control signals. The control signals cause the electroactive polymer actuators to change shape which exerts forces on the tubular member 106 to which they are applied thereby modifying the characteristics of the tubular member 106. As described below, the actuated actuators 114 may change the stiffness and/or shape of the tubular member 106. Upon removal of the electrical control signals, the actuators 114 return to their relaxed configuration and the tubular member 106 also returns to its relaxed configuration including its intrinsic flexibility.

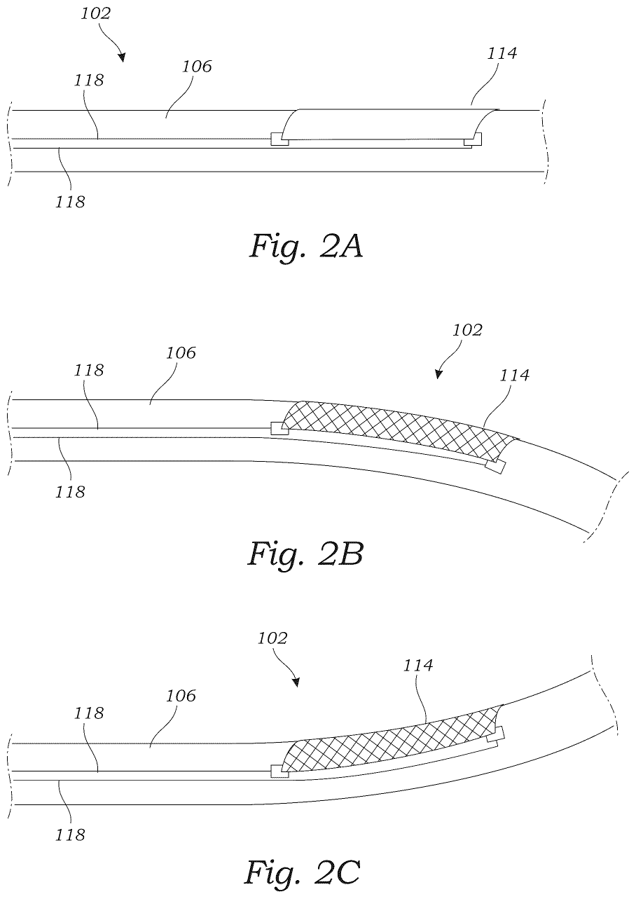

FIGS. 2A-2C illustrate various configurations of electroactive polymer actuators 114 arranged to effect various changes to the configuration of the tubular member 106 on which the electroactive polymer actuators 114 are disposed. FIG. 2A shows an enlarged, side view of the tubular member 106 and one of the electroactive polymer actuators 114 disposed on the tubular member 106. FIG. 2A shows the tubular member 106 and electroactive polymer actuator 114 in a non-actuated configuration (i.e., with no electrical control signal applied). FIG. 2B illustrates an actuated configuration of the electroactive polymer actuator 114 of FIG. 2A, wherein the actuator 114 expands upon actuation by an electrical control signal, and contracts from the expanded state back to the non-actuated configuration upon removal of the electrical control signal. As is shown in FIG. 2B, upon actuation, the electroactive polymer actuator 114 expands thereby causing the segment of the tubular member 106 attached to the electroactive polymer actuator 114 to bend in a convex curve (relative to the side of the tubular member 106 to which the actuator 114 is attached). In addition to bending the tubular member 106, the actuation of the electroactive polymer actuator 114 also stiffens the tubular member 106 in the curved shape.

FIG. 2C illustrates an actuated configuration of the electroactive polymer actuator 114 of FIG. 2A, wherein the actuator 114 contracts upon actuation by an electrical control signal, and expands from the contracted state back to the non-actuated configuration upon removal of the electrical control signal. As is shown in FIG. 2C, upon actuation, the electroactive polymer actuator 114 contracts thereby causing the segment of the tubular member 106 attached to the electroactive polymer actuator 114 to bend in a concave curve (relative to the side of the tubular member 106 to which the actuator 114 is attached). Again, in addition to bending the tubular member 106, the actuation of the electroactive polymer actuator 114 also stiffens the tubular member 106 in the curved shape.

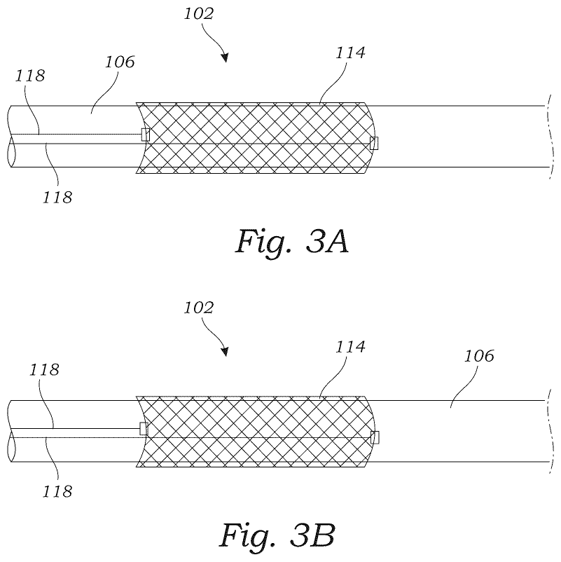

FIG. 3A shows an enlarged, side view of the tubular member 106 and another embodiment of an electroactive polymer actuator 114 disposed on the tubular member 106. In FIG. 3A, the electroactive polymer actuator 114 is configured to stiffen the tubular member 106 in a substantially straight shape. The electroactive polymer actuator 114 is circumferentially disposed on the tubular member 106. Alternatively, a pair of electroactive polymer actuators 114 may be disposed on opposing sides of the tubular member 106 such that they are in tension with one another. FIG. 3A shows the tubular member 106 and electroactive polymer actuator 114 in a non-actuated configuration (i.e., with no electrical control signal applied). FIG. 3B illustrates an actuated configuration of the electroactive polymer actuator 114 of FIG. 3A, wherein the actuator 114 expands upon actuation by an electrical control signal, and expands from the expanded state back to the non-actuated configuration upon removal of the electrical control signal. As is shown in FIG. 3B, upon actuation, the electroactive polymer actuator 114 expands thereby causing the segment of the tubular member 106 attached to the electroactive polymer actuator 114 to expand and stiffen in a straight line. Alternatively, the electroactive polymer actuator 114 of FIG. 3A may be configured to contract upon actuation. Upon actuation, the electroactive polymer actuator 114 contracts resulting in a similar stiffening effect.

The controller 104 is configured to selectively transmit one or more electrical control signals to the electroactive polymer actuators 114. The controller 104 is operably coupled to the electroactive polymer actuators 114 using one or more conductors 118 each having a first end electrically connected to the controller 104 and a second end electrically connected to one or more of the electroactive polymer actuators 114. In one embodiment, there may be a pair of conductors 118 for each electroactive polymer actuator 114. In alternative embodiments, a set of two or more of the electroactive polymer actuators 114 may be connected to the same pair of conductors 118 such that the set of electroactive polymer actuators 114 are actuated by the same electrical control signal from the controller 104. Similarly, the catheter system 100 may be configured with one or more sets each having a plurality of electroactive polymer actuators 114 connected to the same pair of conductors 118.

Accordingly, referring back to FIG. 1, the catheter system 100 includes a catheter 102 which utilizes electroactive polymer actuators 114 to selectively modify the configuration of the catheter 102, such as the shape and/or stiffness of the tubular member 106, using electrical control signal(s). For instance, the steering actuators 114a, 114b, 114c and 114d may be configured to steer the catheter 102 as it is moved within a body lumen. In one embodiment, each of the steering actuators 114a, 114b, 114c and 114d may be configured same or similar to the electroactive polymer actuator 114 of FIG. 2B. In this way, the steering actuators 114a, 114b, 114c and 114d can be selectively actuated using one or more control signals to bend the distal portion 110 of the tubular member 106 in a direction that the user wants to steer the catheter 102 as it is advanced within a body lumen. Each of the steering actuators 114a, 114b, 114c and 114d is independently connected to the controller 104 and controllable by a respective separate control signal from the controller 104. This allows each of the steering actuators 114a, 114b, 114c and 114d to be independently controllable from the other steering actuators 114a, 114b, 114c and 114d. While the exemplary embodiment of FIG. 1 shows four steering actuators 114, any suitable number may be used to provide the desired steering effect. For example, a single steering actuator 114a could be used, and the user would manually manipulate the catheter 102 from the proximal end to rotate the tubular member 106 such that actuating the steering catheter 114a bends the distal portion 110 of the tubular member 106 in the desired steering direction.

The anchoring actuators 114e, 114f and 114g are disposed on an anchoring portion 120 of the tubular member 106. The anchoring actuators 114e, 114f and 114g are configured to be actuated to modify the stiffness and/or shape of the tubular member 106 into an anchoring shape to anchor the anchoring portion in place in a body lumen having a shape corresponding to the anchoring shape. In the exemplary embodiment of FIG. 1, the anchoring actuators 114e and 114g are configured to stiffen and/or shape the tubular member 106 and bias the tubular member 106 into a curved shape, while the anchoring actuator 114f is configured to stiffen the tubular member 106 and bias the tubular member 106 into a substantially straight shape. Accordingly, the anchoring actuators 114e and 114g are same or similar to the electroactive polymer actuator 114 of FIG. 2B. The anchoring actuator 114f is the same or similar to the electroactive polymer actuator 114 depicted in FIGS. 3A-3B. The anchoring actuators 114e, 114f and 114g may be separately connected to the controller 104 such that they can be independently actuated by separate control signals from the controller 104, or they may be connected to the controller 104 as a set such that they can be actuated by a single control signal from the controller 104.

In a non-actuated configuration of the anchoring feature of catheter system 100, the anchoring actuators 114e, 114f and 114g are non-actuated such that the anchoring portion 120 of the tubular member 106 has its intrinsic flexibility. In other words, the anchoring portion 120 is relatively flexible such that it can navigate a relatively tortuous path within a body lumen as the catheter 102 is advanced in the body lumen. In an actuated configuration of the anchoring feature of the catheter system 100, the anchoring actuators 114e, 114f and 114g are actuated by one or more control signals (depending on the configuration of the connection to the controller, as described above). Actuation of the anchoring actuators 114e, 114f and 114g causes the anchoring actuators 114e, 114f and 114g to stiffen and/or shape the anchoring portion 120 of the tubular member 106 into the anchoring shape.

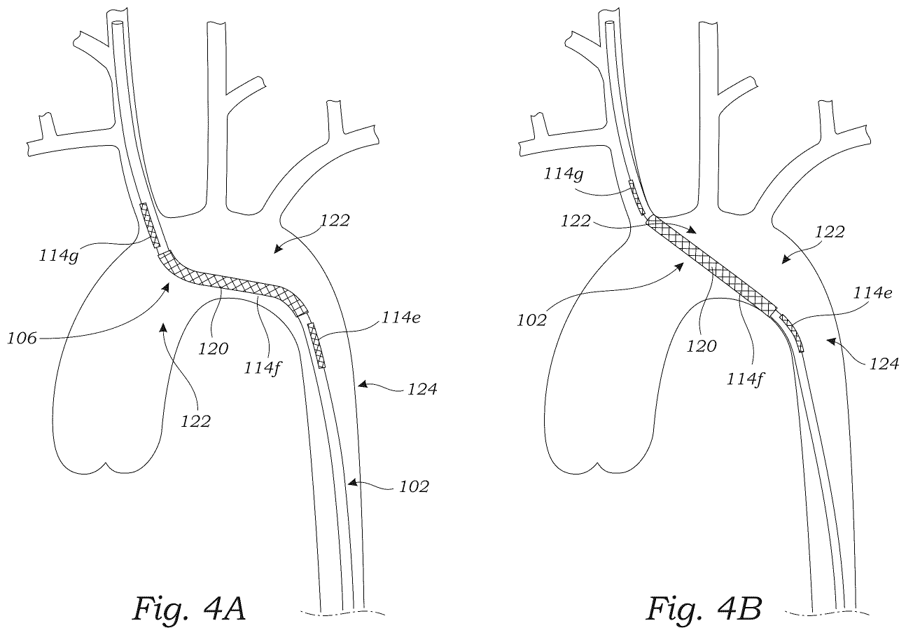

FIGS. 4A-4B illustrate the anchoring feature of the catheter 102 used to anchor or stabilize the catheter 102 in an exemplary body lumen 122. In the illustrated example, the body lumen 122 is an arch traverse 122 within a vascular system 124 comprising a plurality of interconnecting vascular vessels. FIG. 4A depicts the catheter 102 as it is being advanced into the body lumen 122 of the vascular system 124. In FIG. 4A, the catheter 102 is in the non-actuated configuration with the anchoring actuators 114e, 114f and 114g non-actuated, such that the anchoring portion 120 of the tubular member 106 is intrinsically flexible. When the catheter 102 is positioned with the anchoring portion 120 located in the desired location within the body lumen 122, the anchoring actuators 114e, 114f and 114g are actuated using one or more control signals from the controller 104, as shown in FIG. 4B. As shown in FIG. 4B, actuation of the anchoring actuators 114e, 114f and 114g stiffens and shapes the anchoring portion 120 into a shape corresponding to the body lumen 122 such that the anchoring portion 120 wedges into the body lumen 122. This stabilizes and/or anchors the anchoring portion 120 in the body lumen 122, which in turn stabilizes and/or anchors the catheter 102 in place. The anchoring of the catheter 102 prevents the catheter 102 from moving when other instruments, such as other catheters or devices, are inserted and advanced through the catheter 102.

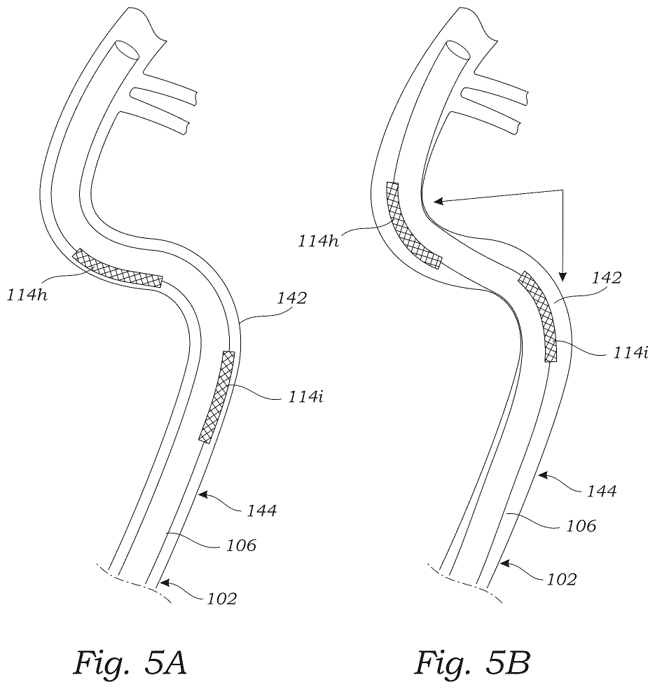

FIGS. 5A-5B illustrate another exemplary embodiment of an anchoring feature on a catheter system 100. The anchoring feature shown in FIGS. 5A-5B is similar to the anchoring feature depicted in FIGS. 4A-4B, except that it is configured to be used in a specific curved body lumen 124 having an S-shaped curve (similar to the shape of a typical petrous carotid artery). The catheter 140 of FIGS. 5A-5B does not require a straight shaped anchoring actuator 114f as in the catheter 102 of FIGS. 4A-4B. Instead, the catheter 140 has an anchoring actuator 114h and an anchoring actuator 114i which are configured to curve in opposite directions to form an anchoring shape which corresponds to the S-curve shape of the body lumen 142. FIG. 5A depicts the catheter 140 as it is being advanced into the body lumen 142 of the vascular system 144. In FIG. 5A, the catheter 140 is in the non-actuated configuration with the anchoring actuators 114h and 114i non-actuated, such that the anchoring portion 120 of the tubular member 106 is intrinsically flexible. When the catheter 140 is positioned with the anchoring portion 120 located in the desired location within the body lumen 142, the anchoring actuators 114h and 114i are actuated using one or more control signals from the controller 104, as shown in FIG. 5B. As shown in FIG. 5B, actuation of the anchoring actuators 114h and 114i stiffens and shapes the anchoring portion 120 into an S-shape which anchors the anchoring portion 120 into a shape corresponding to the body lumen 142. This stabilizes/or anchors the anchoring portion 120 in the body lumen 142, which in turn stabilizes and/or anchors the catheter 140 in place. The anchoring of the catheter 102 prevents the catheter 140 from moving when other instruments, such as other catheters or devices, are inserted and advanced through the catheter 102.

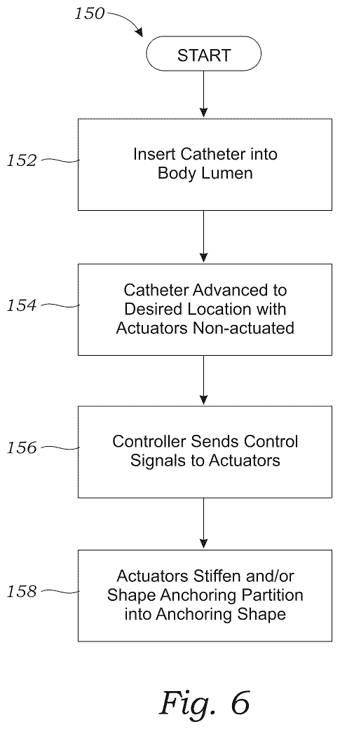

Turning to FIG. 6, a flow chart depicting a method 150 of using the catheter system 100 having either of the catheters 102 or 140 is shown. The method of using the catheter system 100 with either of the catheters 102 or 140 is substantially the same, and therefore, the method 150 will be described for the catheter system having catheter 102. At step 152, the distal end 110 of the catheter 102 is inserted into the body lumen with the anchoring actuators 114e, 114f and 114g non-actuated, such that the anchoring portion 120 of the tubular member 106 has its intrinsic flexibility. At step 154, the catheter 102 is advanced into the body lumen with the anchoring actuators 114e, 114f and 114g non-actuated to position the catheter 102 in a desired location with the anchoring portion positioned in the body lumen 122. At step 156, the controller 104 sends one or more control signals to the anchoring actuators 114e, 114f and 114g thereby actuating the anchoring actuators 114e, 114f and 114g. At step 158, the actuation of the anchoring actuators 114e, 114f and 114g stiffen and/or shape the anchoring portion 120 into the anchoring shape which anchors the anchoring portion 120 in place in the body lumen 122.

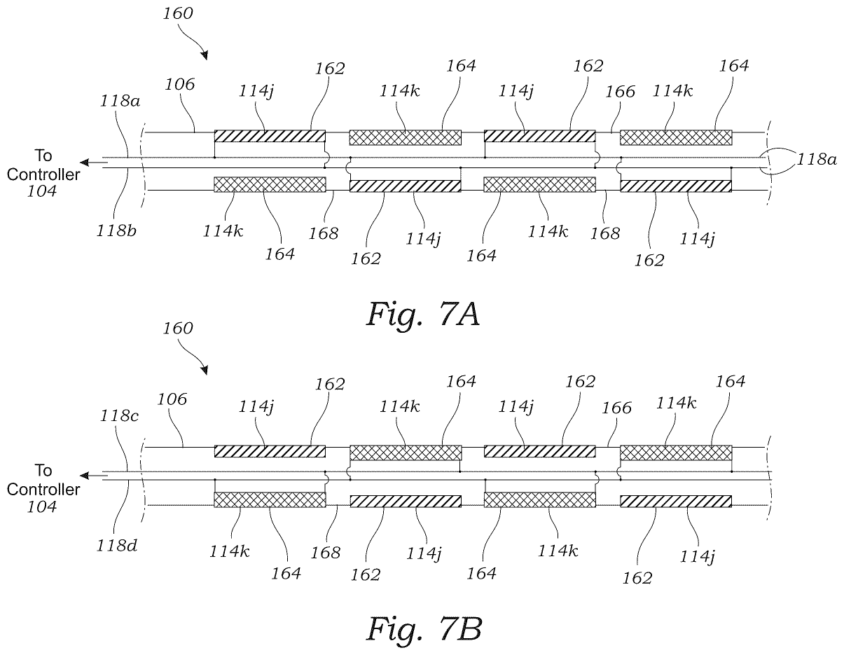

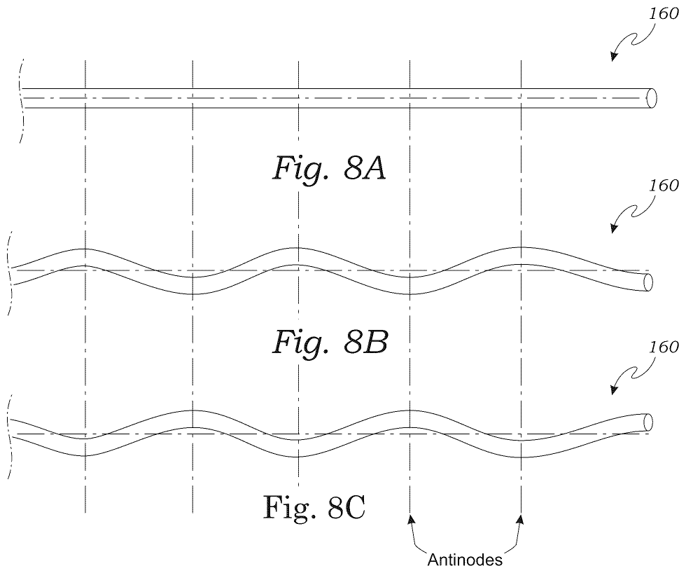

Turning now to FIGS. 7A-7B and 8A-8C, another exemplary embodiment of a catheter 160 having actuators 114 which can be used in the catheter system 100 of FIG. 1 is illustrated. Moreover, the catheter 160 may include any one or more of the features and aspects of the catheter 102 as shown in the drawings and described herein. The catheter 160 is configured to utilize the actuators 114 to dynamically cycle the shape of the catheter 160 to reduce static friction of the catheter 160 as it is moved longitudinally in a body lumen. As shown in FIGS. 7A-7B, the catheter 160 includes an elongated, flexible, tubular member 106 and a plurality of actuators 114 disposed on the tubular member 106. The actuators 114 are configured and arranged to modify the shape of the tubular member 106 into a substantially sinusoidal shape and to dynamically cycle the sinusoidal shape back and forth from 0° out of phase (as shown in FIG. 8B) to 180° out of phase (as shown in FIG. 8C). FIGS. 7A-7B, and 8A-8C show a representative segment of the catheter 160, with the understanding that the actuators 114j, 114k may be disposed on any suitable length of the tubular member 106, including being disposed on substantially the entire length of the tubular member 106 extending from the proximal end 108 to the distal end 110 of the tubular member 106, or they may be disposed on substantially an entire insertable length of the tubular member 106.

FIGS. 7A and 7B illustrate one exemplary arrangement of the actuators 114 configured to form the tubular member 106 into a sinusoidal shape 0° out of phase and a sinusoidal shape 180° out of phase. The plurality of actuators 114 includes a first set 162 of actuators 114j and a second set 164 of actuators 114k. On a first side 166 (radially) of the tubular member 106, the actuators 114j of the first set 162 are disposed longitudinally along the tubular member 106 and alternating with actuators 114k of the second set 164 disposed longitudinally along the tubular member 106 on the first side 166. Similarly, on a second side 168 (radially) of the tubular member 106 radially opposite the first side 166, the actuators 114j of the first set 162 are disposed longitudinally along the tubular member 106 and alternating with actuators 114k of the second set 164 disposed longitudinally along the tubular member 106 on the second side 168.

As shown in FIGS. 7A-7B, the actuators 114j of the first set 162 are arranged such that the actuators 114j on the first side 166 are longitudinally offset from the actuators 114j on the second side 168. Similarly, the actuators 114k of the second set 164 are arranged such that the actuators 114k on the first side 166 are longitudinally offset from the actuators 114k on the second side 168. As also shown in FIGS. 7A-7B, this arrangement results in the actuators 114j of the first set 162 on the first side 166 oppose the actuators 114k of the second set 168 on the second side 168, and the actuators 114j of the first set 162 on the second side 168 oppose the actuators 114k of the second set 164 on the first side 166, and vice versa. The configuration of the actuators 114 is such that actuation of the first set 162 of actuators 114j modifies the shape of the tubular member 106 into the substantially sinusoidal shape 0° out of phase, as shown in FIG. 8B, and actuation of the second set 164 of actuators 114k modifies the shape of the tubular member 106 into the sinusoidal shape 180 out of phase, as shown in FIG. 8C.

Referring to FIG. 7A, when the actuators comprise an electroactive polymer, the first set 162 of electroactive polymer actuators 114j are electrically connected to the controller 104 via a first pair of electrical connections 118a, 118b such that all of the electroactive polymer actuators 114j in the first set 162 are actuated by a single first electrical control signal from the controller 104. One connection 118a of the pair electrical connections is connected to a respective first end of each of the electroactive polymer actuators 114j, and the other connection 118b is connected to a respective second end of each of the electroactive polymer actuators 114j. The electrical connections 118a, 118b may each comprise a respective wire disposed on the tubular member 106 and extending from the distal end 110 to the proximal end 108 of the catheter 160 and to a connection to the controller 104. Instead of wires, the electrical connections 118a, 118b may comprise any other suitable electrical conductor such as a conductive material coated or printed on the tubular member 106, etc.

Similarly, as shown in FIG. 7B, the second set 164 of electroactive polymer actuators 114k are connected to the controller 104 via a second pair of electrical connections 118c, 118d such that all of the electroactive polymer actuators 114k in the second set 164 are actuated by a single second electrical control signal from the controller 104. The second pair of electrical connections 118c, 118d are electrically insulated/separated from the first pair of electrical connections 118a, 118b. One connection 118c of the pair electrical connections is connected to a respective first end of each of the electroactive polymer actuators 114k, and the other connection 118d is connected to a respective second end of each of the electroactive polymer actuators 114k. The electrical connections 118c, 118d may each comprise a respective wire disposed on the tubular member 106 and extending from the distal end 110 to the proximal end 108 of the catheter 160 and to a connection to the controller 104. Instead of wires, the electrical connections 118c, 118d may comprise any other suitable electrical conductor such as a conductive material coated or printed on the tubular member 106, etc.

As depicted in FIGS. 8A-8C, the plurality of actuators 114 on the catheter 160 may be actuated dynamically to cycle the tubular member 106 back and forth between a substantially sinusoidal shape 0° out of phase and a substantially sinusoidal shape 180° out of phase by alternating actuation of the first set 162 and the second set 164 using the first signal and second signal. FIG. 8A shows the catheter 160 with both the first set 162 and the second set 164 non-actuated such that the tubular member 106 has its inherent flexibility and relaxed shape which typically is the shape of the path of the body lumen in which the catheter 160 is inserted. As shown in FIG. 8B, upon actuation of the first set 162 of actuators 114j by a first control signal from the controller 104 (with the second set 164 remaining non-actuated), the actuators 114j form the tubular member 106 into a substantially sinusoidal shape 0° out of phase. As shown in FIG. 8C, upon actuation of the second set 164 of actuators 114k by a second control signal from the controller 104 (and the first set 162 being de-actuated by turning off the first control signal), the actuators 114k form the tubular member 106 into a substantially sinusoidal shape 180° out of phase. The controller 104 is configured to modulate the first and second 1 control signals in an alternating pattern to dynamically cycle the actuation of the first set 162 of actuators 114j and the second set 164 actuators 114k to dynamically cycle the tubular member 106 back and forth between the substantially sinusoidal shape 0° out of phase and a substantially sinusoidal shape 180° out of phase.

The catheter system 100 having the catheter 160 is configured to dynamically cycle the sinusoidal shape of the tubular member 106 at an amplitude and frequency which reduces static friction of the tubular member 106 as it is moved longitudinally with a body lumen. The amplitude and frequency may be set to be a minimum needed to reduce the static friction, and which does not irritate the walls of the body lumen (such as blood vessels) or cause spasm. For instance, exemplary frequencies are in the range of from 1 Hz to 10 Hz (cycles per second), and exemplary amplitude is within a range of from 0.1 mm to 1.0 mm (this compares with exemplary amplitudes for the movement of the steering actuators 114a-114d in the range of 2.0 mm-10.0 mm).

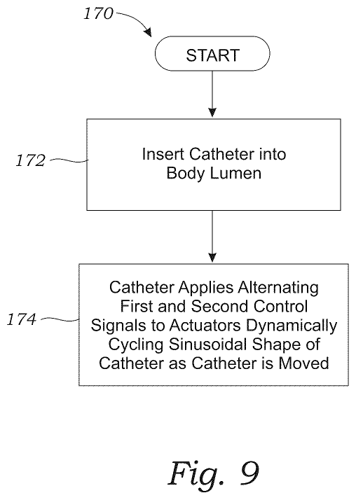

Referring to FIG. 9, a flow chart showing a method 170 of using the catheter system 100 implementing the catheter 160 is shown. The method 170 includes a step 172, in which the distal end 110 of the catheter 160 is inserted into a body lumen. The catheter 160 may be inserted into the body lumen with the actuators 114 non-actuated, or with the actuators 114 being dynamically actuated to reduce static friction as the catheter 160 is being inserted. It is contemplated that the catheter 160 will be initially inserted into the body lumen for at least a short length in order to have a more stable catheter 160 as it is first inserted. At step 174, the controller applies the alternating first and second electrical control signals thereby dynamically cycling the tubular member 106 back and forth between the substantially sinusoidal shape 0° out of phase and a substantially sinusoidal shape 180° out of phase as the catheter 102 is moved longitudinally within the body lumen. The cycling sinusoidal motion of the tubular member 106 breaks the static friction of the tubular member 106 making the longitudinal movement easier (i.e., requiring less force on the catheter 160) and smoother.

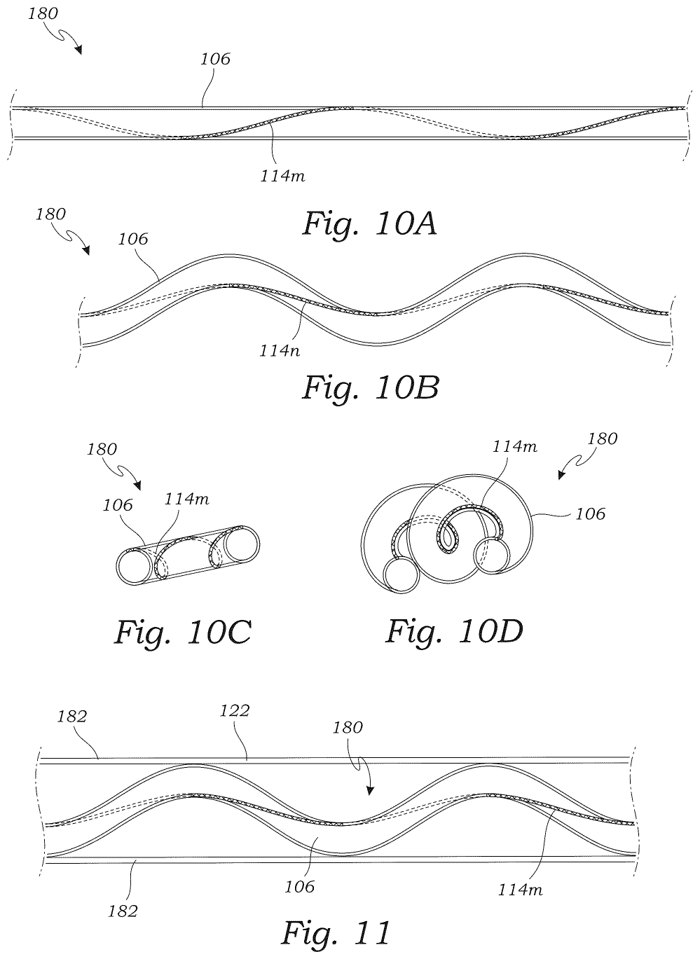

FIGS. 10A-10D and 11 illustrate another exemplary embodiment of a catheter 180 having actuators 114 which can be used in the catheter system 100 of FIG. 1. In addition, the catheter 180 may include any one or more of the features and aspects of the catheter 102 as shown in the drawings and described herein. The catheter 180 utilizes a single helically arranged actuator 114m configured to shape the tubular member 106 into a helical shape which makes circumferential contact with the wall 182 of a body lumen 122 to stabilize the catheter 180 within the body lumen 122. As shown in FIG. 10A, the catheter 180 includes a tubular member 106. The actuator 114m is disposed on the tubular member 106 in a helical path longitudinally along the tubular member 106. FIGS. 10A-10D, and 11 show a representative segment of the catheter 180, with the understanding that the actuator 114m may be disposed on any suitable length of the tubular member 106, including being disposed on substantially the entire length of the tubular member 106 extending from the proximal end 108 to the distal end 110 of the tubular member 106, or they may be disposed on substantially an entire insertable length of the tubular member 106. As shown in FIGS. 10A and 10C, when the actuator 114 in non-actuated, the tubular member 106 has its intrinsic flexibility and its relaxed shape (typically, it takes on the shape of the path of the body lumen 122 in which the catheter is inserted). In FIGS. 10A and 10C, the non-actuated catheter 180 is substantially straight. As shown in FIGS. 10B and 10D, upon actuation of the actuator 114m, the actuator 114m transitions the shape of the tubular member 106 into a substantially helical shape. The actuator 114m contracts (i.e., is in tension), causing the tubular member 106 to form into a substantially helical shape such that the actuator 114m is on the inner radius of the helically shaped tubular member 106 (as best shown in FIG. 10D).

As shown in FIGS. 1, 2A-3B, and 7A-7B, when the actuator comprises an electroactive polymer, the catheter 180 also has a pair of electrical connections 118 each having one end connected to the electroactive polymer actuator 114m, and one end connected to the controller 104. When the electroactive polymer actuator 114m is non-actuated, the tubular member 106 has its intrinsic flexibility and its relaxed shape (which typically is the shape of the path of the body lumen 122 in which the catheter 180 is inserted), as depicted in FIGS. 10A and 10C.

As shown in FIGS. 10B and 10D, upon actuation of the electroactive polymer actuator 114m by an electrical control signal from the controller 104, the electroactive polymer actuator 114m transitions the shape of the tubular member 106 into a substantially helical shape. The actuator 114m contracts (i.e., is in tension), causing the tubular member 106 to form into a substantially helical shape such that the actuator 114m is on the inner radius of the helically shaped tubular member 106.

The singular actuator 114m may comprise any type of elongate linearly acting contracting or expanding actuator. If the actuator 114m is a contracting actuator, upon contraction the actuator will force a shortening of the side of the tubular member 106 where it is helically disposed causing the catheter 180 to assume a helical shape with the actuator 114m biased to the interior of the helical shape. If the actuator is an expanding actuator, upon expansion the actuator will force a lengthening of the side of the tubular member 106 where it is helically disposed causing the catheter 180 to assume a helical shape with the actuator 114m biased to the exterior of the helical shape.

As shown in FIG. 11, the catheter 180 is configured such that the substantially helical shaped tubular member 106 makes circumferential contact (contact around the full circumference) of the wall 182 of the body lumen 122. The circumferential contact of tubular member 106 with the wall 182 stabilizes the catheter 180 in the body lumen 122. For example, the catheter 106 is prevented from moving radially, and also longitudinally which prevents longitudinal movement of the catheter 180 relative to the body lumen 122, while instruments and other catheters are navigated and/or manipulated through the tubular member 106.

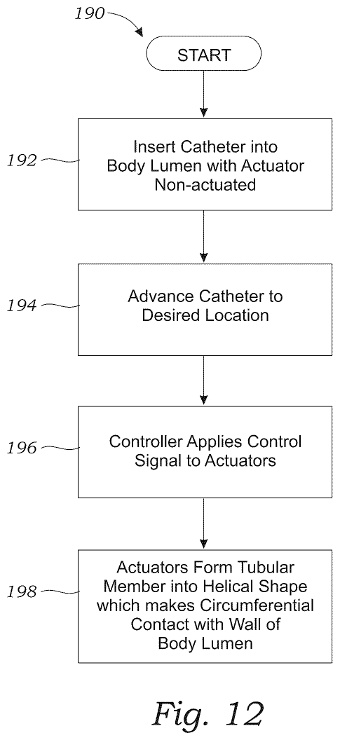

Turning to FIG. 12, a flow chart illustrating a method 190 of using the catheter system 100 implementing the catheter 180 is shown. The method 190 includes a step 192 of inserting the catheter 180 into a body lumen 122 with the electroactive polymer actuator 114m non-actuated such that the tubular member 106 has its intrinsic flexibility and relaxed shape. At step 194, the catheter 180 is advanced into the body lumen to a desired location within the body lumen 122. At step 196, the controller 104 applies an electrical control signal to the electroactive polymer actuator 114m. At step 198, the controller 104 actuates the electroactive polymer actuator 114m which transitions the shape of the tubular member 106 into the substantially helical shape such that the tubular member 106 makes circumferential contact with the wall 182 of the body lumen 122. The circumferential contact stabilizes and/or anchors the catheter 180 in the body lumen 122.

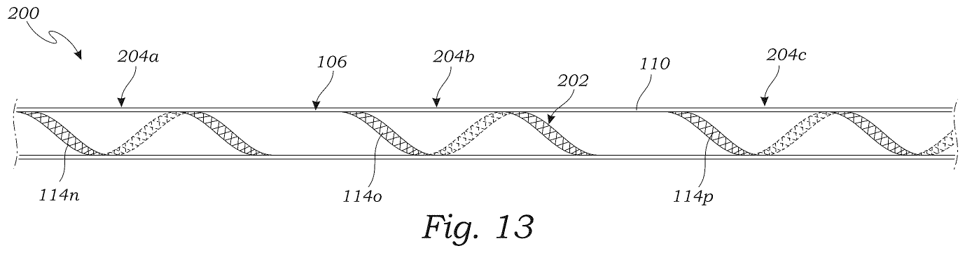

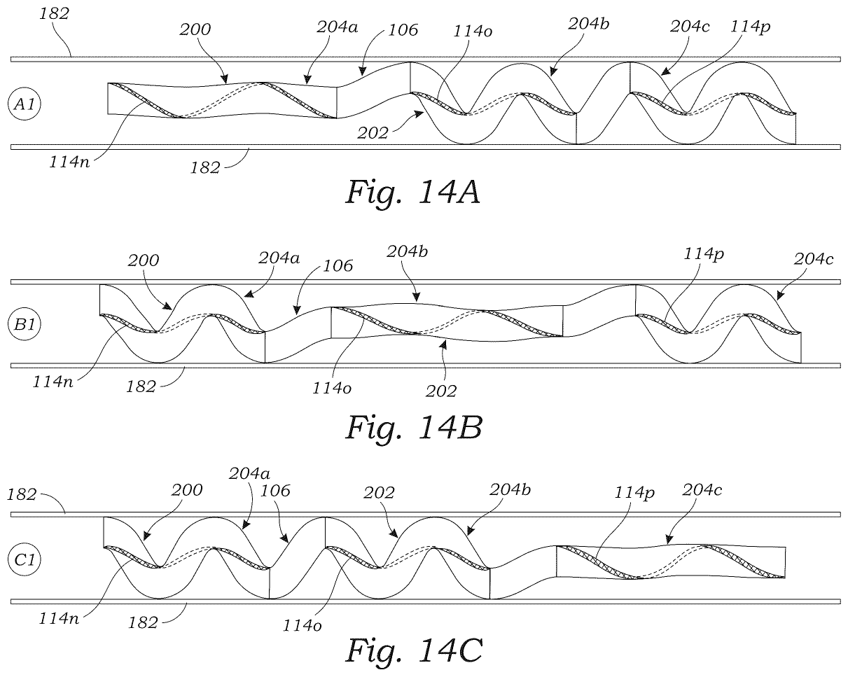

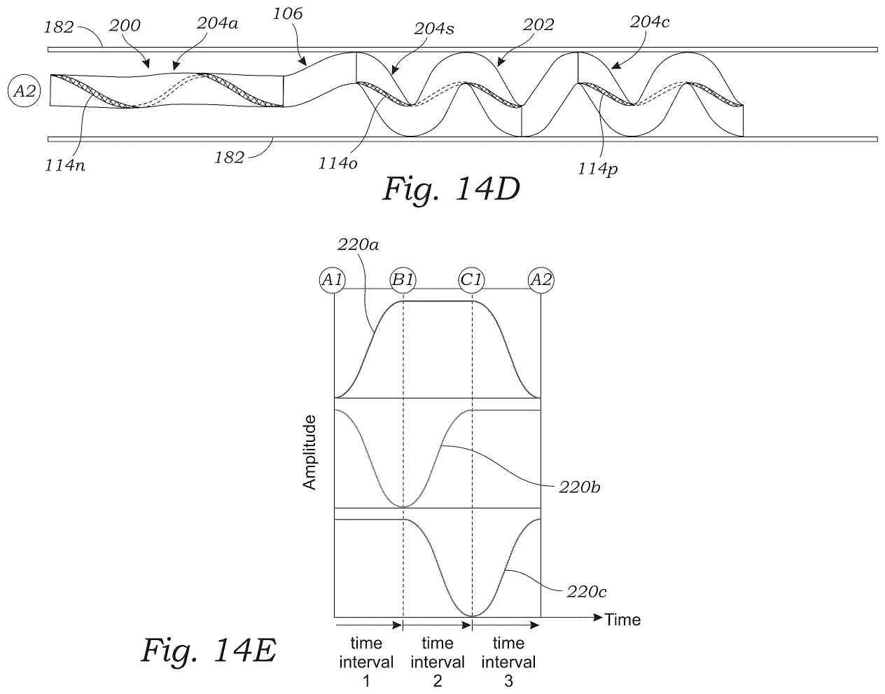

FIGS. 13 and 14A-14E illustrate another exemplary embodiment of a catheter 200 having actuators 114 which can be used in the catheter system 100 of FIG. 1. In addition, the catheter 200 may include any one or more of the features and aspects of the catheter 102 as shown in the drawings and described herein. The catheter 200 utilizes a motive actuator 202 comprising a plurality of helically arranged actuators 114n, 114o, 114p, etc. to create a motive force to move or assist in the movement of the catheter 200 longitudinally within a body lumen 122. The illustrated embodiment of the catheter 200 includes a motive actuator 202 having three helical actuators 114n, 114o, and 114p, but could utilize any suitable number of helical actuators 114.

Like the other catheters described herein, the catheter 200 includes an elongated, flexible tubular member 106. The tubular member 106 has a plurality of longitudinal segments or portions 204, one segment 200 for each helical actuator 114. In the illustrated embodiment, the tubular member 106 has three segments 204a, 204b and 204c. The segments 204a, 204b and 204c are longitudinally located apart from each other, such as a first segment 204a, a second segment 204b distal of the first segment 204a, and a third segment 204c distal of the second segment 204b. The three segments 204a, 204b and 204c may be positioned at the distal portion 110 of the tubular member 106. In this way, the motive actuator 202 will typically be inserted into a body lumen 122 first along with the distal portion 110 of the catheter 200 and then the motive actuator 202 can push or pull the catheter 200 into the body lumen 122 to advance the catheter 200 to a desired location in the body lumen 122.

Each helical actuator 114n, 114o, and 114p is disposed on a respective segment 204a, 204b and 204c in a helical path along the respective segment 204a, 204b and 204c such that actuation of the respective actuator 114n, 114o, and 114p using a respective control signal transitions a shape of the respective segment 204a, 204b and 204c into a substantially helical shape, as shown in FIGS. 14A-14D. FIG. 14A shows actuator 114n in a non-actuated state and actuators 114o and 114p being actuated to form segments 204b and 204c into respective helical shapes. FIG. 14B shows actuator 114o in a non-actuated state and actuators 114n and 114p being actuated to form segments 204a and 204c into respective helical shapes. FIG. 14C shows actuator 114p in a non-actuated state and actuator 114n and 114o being actuated to form segments 204a and 204b into respective helical shapes.

Each respective actuator 114n, 114o, and 114p is configured such that modulated actuation of the actuators 114n, 114o, and 114p creates a motive force between the tubular member 106 and the wall 182 of the body lumen 122 which moves the tubular member 106 longitudinally within the body lumen 122.

The outer surface of each of the segments 204a, 204b and 204c may have helical shaped undulations, ribs or other structure to facilitate the movement of the tubular member 106 in response to the modulated actuation. The undulations, ribs or other structure can increase the friction between the tubular member 106 and the walls 182 of the body lumen 122.

In one way, the modulated actuation of the polymer actuators 114n, 114o, and 114p causes the tubular member 106 to move in a worm crawler-like motion, as depicted in FIGS. 14A-14E. To produce the worm crawler-like motion, each of the actuators 114n, 114o, and 114p is actuated in a modulated fashion which is out of phase from the other two actuators. In the exemplary embodiment depicted in FIGS. 14A-14E, the modulated actuation of each actuator 114 is 120° out phase with the other actuators 114. FIG. 14E illustrates an example of the modulated actuation state/signal of the actuators 114n, 114o, and 114p, over time, to produce a crawler-like motive force to move the tubular member 106. In the graph of FIG. 14E, the actuation line 220a corresponds to the actuation state of actuator 114n, the actuation line 220b corresponds to the actuation state of actuator 114o, and the actuation line 220c corresponds to the actuation state of actuator 114p. The times A1, B1, C1 and A2 in FIG. 14E correspond to the like references in FIGS. 14A-14D.

The operation of the catheter 200 to move within a body lumen 122 having a wall 182, with reference to FIGS. 14A-14E will now be described. As shown in FIG. 14E, at time A1, actuator 114n is non-actuated, actuator 114o is fully actuated and actuator 114p is fully actuated. FIG. 14A shows the configuration of the catheter 200 corresponding to the actuation state at time A1, in which non-actuated actuator 114 forms segment 204a into a substantially straight shape, fully actuated actuator 114o form segment 204b into a helical shape which contacts the wall 182, and fully actuated actuator 114o form segment 204b into a helical shape which contacts the wall 182. As shown in FIG. 14E, as the modulated actuation proceeds from time A1 to time B1 (“time interval 1”), actuator 114n is gradually actuated, actuator 114o is gradually de-actuated, and actuator 114p remains fully actuated. During time interval 1, actuator 114n forms segment 204a into a helical shape, actuator 114o transitions segment 204b from a helical shape to a substantially straight shape, and actuator 114p maintains segment 204c in a helical shape. During time interval 1, segment 204c anchors the trailing end of the motive actuator 202 to the wall 182, and the extension of segment 204b pushes segment 204a to the left.

As shown in FIG. 14E, as the modulated actuation proceeds from time B1 to time C1 “time interval 2”), actuator 114n remains actuated, actuator 114o is gradually actuated, and actuator 114p is gradually de-actuated. During time interval 2, actuator 114n maintains segment 204a in a helical shape, actuator 114o transitions segment 204b from a substantially straight shape to a helical shape, and actuator 114p transitions segment 204c from a helical shape to a substantially straight shape. During time interval 2, segment 204a anchors the leading end of the motive actuator 202 to the wall 182, segment 204c releases from the wall 182, and the contraction of segment 204b as it forms into a helical shape pulls segment 204c to the left.

As shown in FIG. 14E, as the modulated actuation proceeds from time C1 to time A2 “time interval 3”), actuator 114n is gradually de-actuated, actuator 114o remains fully actuated, and actuator 114p is gradually actuated. During time interval 3, actuator 114n transitions segment 204a from a helical shape to a substantially straight shape, actuator 114o maintains segment 204b in a helical shape, and actuator 114p transitions segment 204c from a substantially straight shape to a helical shape. During time interval 3, segment 204b anchors the middle of the motive actuator 202 to the wall 182, segment 204a releases the leading end of the motive actuator 202 from the wall 182 and extends to the left, and segment 204c contracts as it forms into a helical shape in which it again anchors the trailing end of the motive actuator 202 to the wall 182. At time A2, the motive actuator 202 is in the same configuration as at time A1, and the modulated actuation can be repeated to continue moving the catheter 200. Of course, the modulated actuation can be reversed in order to produce a motive force in the opposite direction, thereby moving the catheter 200 to the right.

The degree of phase shift between the actuators 114 may be different depending on the number of actuators 114, and other considerations like the design of the motion, to produce the desired motive force. In addition, the actuators 114 in the catheter 200 may be any suitable actuator, including electroactive polymer actuator, or other types of actuators disclosed herein. In the case of electroactive polymer actuators 114, the catheter 200 also has a pair of electrical connections 118 to each of electroactive polymer actuators 114, allowing independent actuation of each actuator 114. The controller 104 is configured to provide the modulated electrical actuation signals to each of the actuators 114n, 114o and 114p according to the modulation pattern to produce the motive force.

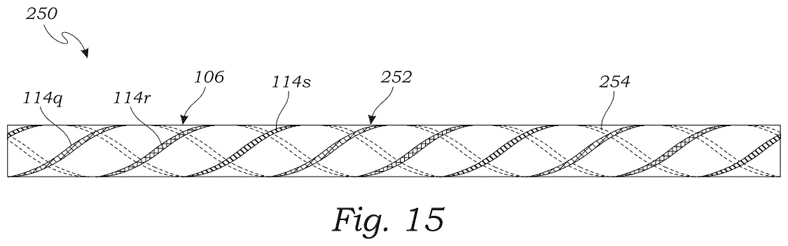

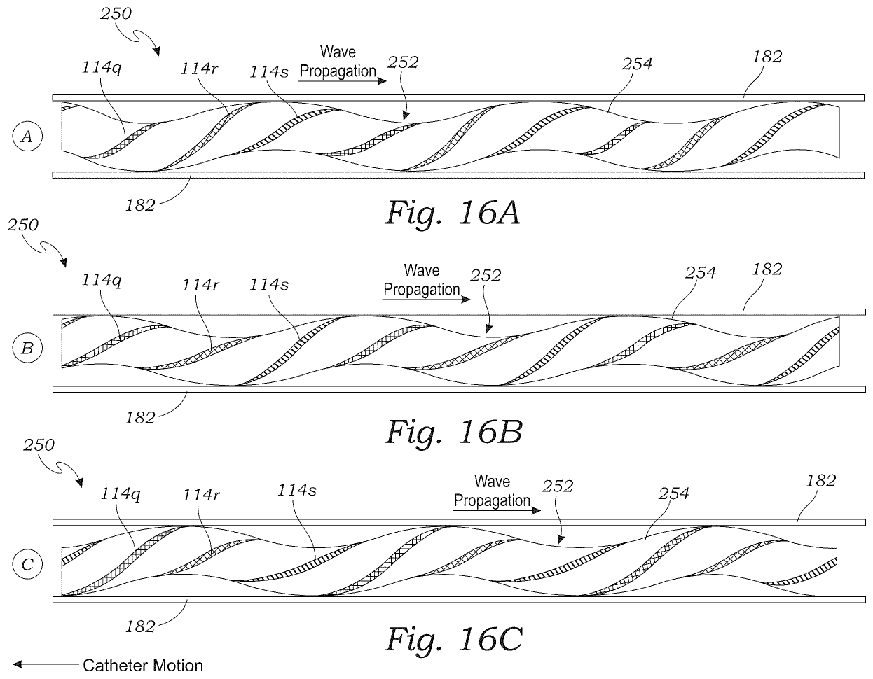

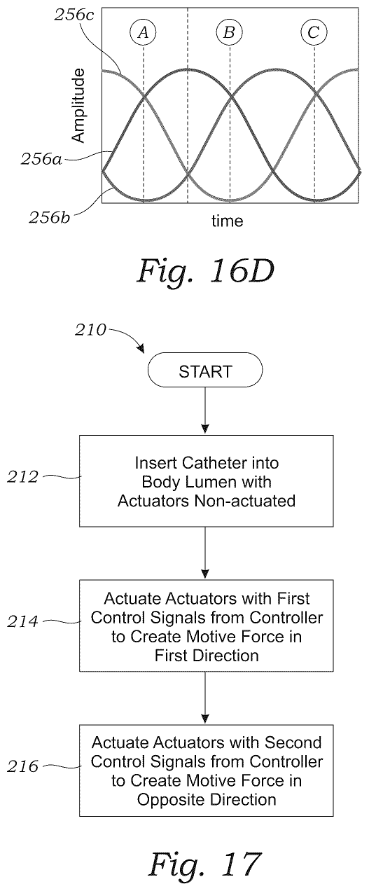

FIGS. 15 and 16A-16D illustrate another exemplary embodiment of a catheter 250 having a motive actuator 252 for creating a motive force to move or assist in the movement of the catheter longitudinally within a body lumen 122. Like the catheter 200, the catheter 250 has actuators 114 and can be used in the catheter system 100 of FIG. 1. In addition, the catheter 250 may include any one or more of the features and aspects of the catheter 102 as shown in the drawings and described herein. The motive actuator 250 comprises a plurality of helically arranged actuators 114q, 114r, 114s, etc. to create a motive force to move the catheter 250 longitudinally within a body lumen 122. The illustrated embodiment of the catheter 250 includes a motive actuator 252 having three helical actuators 114q, 114r, and 114s, but could utilize any suitable number of helical actuators 114.

Like the other catheters described herein, the catheter 250 includes an elongated, flexible tubular member 106.

Each helical actuator 114q, 114r and 114s is disposed on the tubular member 106 in a helical path along the longitudinal length of the tubular member 106, with each helical path of an actuator 114 spaced apart, i.e., offset, longitudinally from the helical paths of the other actuators 114. As illustrated in FIGS. 15 and 16A-16C, the order of the actuators 114 goes from left to right in the order of 114q, 114r, 114s, with the actuators 114q, 114r, 114s arranged in parallel along the tubular member. The motive actuator 252 comprised of the helical actuators 114q, 114r and 114s may extend along a portion 254 of the tubular member 106, or along the entire length of the tubular member 106, or along at least a percentage of the length of the tubular member 106 (e.g., 90%, or 80%, or 70%, or 60% or 50%), or along some other suitable length of the tubular member. For example, the motive actuator 252 may be disposed on a portion 254 of the distal part of the catheter 250. In this way, the motive actuator 252 can be inserted into a body lumen 122 first along with the distal portion 110 of the catheter 250 and then the motive actuator 252 can be used to push or pull the catheter 250 into the body lumen 122 and to advance or retract the catheter 200 to a desired location in the body lumen 122.

Each helical actuator 114q, 114r, and 114s is disposed on the tubular member 106 and is configured such that actuation of the respective actuator 114q, 114r, and 114s using a respective control signal forms the tubular member 106 into a substantially helical shape, as shown in FIGS. 16A-16C. Due to the offset location of each of the actuators 114q, 114r, and 114s, the helical shape of the tubular member 106 formed by each of the actuators 114q, 114r, and 114s is also offset. Accordingly, a modulated actuation of the actuators 114q, 114r, and 114s can form the tubular member 106 into a helical shape which propagates in a wave-form along the longitudinal length of the tubular member 106. The wave propagation of the helical shape along the longitudinal length of the tubular member 106 in a first direction (e.g., to the right), creates an opposite motive force (e.g., to the left) of the tubular member 106 against the wall 182 of the body lumen 122 which causes the catheter 250 to move in the direction of the motive force.

The outer surface of the tubular member 106 of the catheter 250 may have helical shaped undulations, ribs or other structure to facilitate the movement of the tubular member 106 in response to the modulated actuation. The undulations, ribs or other structure can increase the friction between the tubular member 106 and the walls 182 of the body lumen 122.

FIGS. 16A-16D illustrate one example of a modulated actuation of the motive actuator 250 for creating a motive force to move the catheter 250. FIG. 16D shows the respective actuation states/signals for each of the actuators 114q, 114r and 114s, over time, to produce a helical shape which propagates in a wave-form from left to right. The actuation line 256a corresponds to the actuation state of actuator 114q, the actuation line 256b corresponds to the actuation state of actuator 114r, and the actuation line 256c corresponds to the actuation state of actuator 114s. The times A, B, and C in FIG. 16D correspond to the like references in FIGS. 16A-16C. The actuation state/signals 256 are modulated in a sine-wave form, in which the sine-wave actuation state/signal for each actuator 114 is 120° out phase with the other actuators 114. In other words, the sine-wave actuation state/signal 256b for the actuator 114r is 120° out of phase from the actuation state/signal 256a for the actuator 114q, the sine-wave actuation state/signal 256c for the actuator 114s is 120° out of phase from the actuation state/signal 256b for the actuator 114r, and the sine-wave actuation state/signal 256a for the actuator 114q is 120° out of phase from the actuation state/signal 256c for the actuator 114s.

As shown in FIG. 16A, at time A of FIG. 16D, the actuators 114q and 114s are actuated and the actuator 114r is non-actuated. This forms the tubular member 106 into a helical shape in which the actuator 114r and part of the tubular member 106 attached to the actuator 114r are on the outer radius of the helical shape, while the actuators 114q and 114s and respective parts of the tubular member 106 are on the inside radius of the helical shape. As shown in FIG. 16B, at time B of FIG. 16D, the actuators 114q and 114r are actuated and the actuator 114s is non-actuated. This forms the tubular member 106 into a helical shape in which the actuator 114s and part of the tubular member 106 attached to the actuator 114s are on the outer radius of the helical shape, while the actuators 114q and 114r and respective parts of the tubular member 106 are on the inside radius of the helical shape. As shown in FIG. 16C, at time C of FIG. 16D, the actuators 114r and 114s are actuated and the actuator 114q is non-actuated. This forms the tubular member 106 into a helical shape in which the actuator 114q and part of the tubular member 106 attached to the actuator 114q are on the outer radius of the helical shape, while the actuators 114r and 114s and respective parts of the tubular member 106 are on the inside radius of the helical shape.

By modulating the respective actuation signals 256a, 256b and 256, as shown in FIG. 16D, the helical shape of the tubular member 106 propagates in a wave-form along the longitudinal length of the tubular member 106 in a first direction (e.g., to the right), creating an opposite motive force (e.g., to the left) of the tubular member 106 against the wall 182 of the body lumen 122 which causes the catheter 250 to move in the direction of the motive force. The modulated actuation pattern can be reversed in order to produce a motive force in the opposite direction, thereby moving the catheter 250 to the right. For instance, the modulated actuation of the actuators 114q, 114r, and 114s may cause the tubular member 106 to move in a rotating screw motion which creates a motive force to move the catheter 250 longitudinally.

The degree of phase shift between the actuators 114 in the motive actuator 252 may be different depending on the number of actuators 114, and other considerations like the design of the motion, to produce the desired motive force. In addition, the actuators 114 in the catheter 250 may be any suitable actuator, including electroactive polymer actuator, or other types of actuators disclosed herein. In the case of electroactive polymer actuators 114, the catheter 250 also has a pair of electrical connections 118 to each of electroactive polymer actuators 114, allowing independent actuation of each actuator 114. The controller 104 is configured to provide the modulated electrical actuation signals to each of the actuators 114q, 114r and 114s according to the modulation pattern to produce the motive force.

Turning to FIG. 17, a flow chart illustrating a method 210 of using the catheter system 100 implementing either of the catheters 200 and 250 is shown. The method 210 includes a step 212 of inserting the catheter 200 into a body lumen 122 with the actuators 114n, 114o and 114p non-actuated such that the tubular member 106 has its intrinsic flexibility and relaxed shape. At step 214, the actuators 114n, 114o and 114p are actuated using a first pattern of respective modulated control signals to each actuator 114n, 114o and 114p. The controller 104 provides the control signals which may comprise various forms of energy including electrical, mechanical, hydraulic or other means in which an energy signal may be transferred. The respective modulated control signals are out of phase with each other (e.g., 120° out of phase). The modulated actuation transitions the respective segments 204a, 204b and 204c into helical shapes in a first modulated pattern thereby creating a motive force between the tubular member 106 and the wall 182 of the body lumen 122. At step 216, actuators 114n, 114o and 114p are actuated using a second pattern of modulated control signals (e.g., the second pattern may be a reverse of the first pattern) which withdraws the tubular member 106 (i.e., moves the tubular member 106 in the opposite direction of advancing).

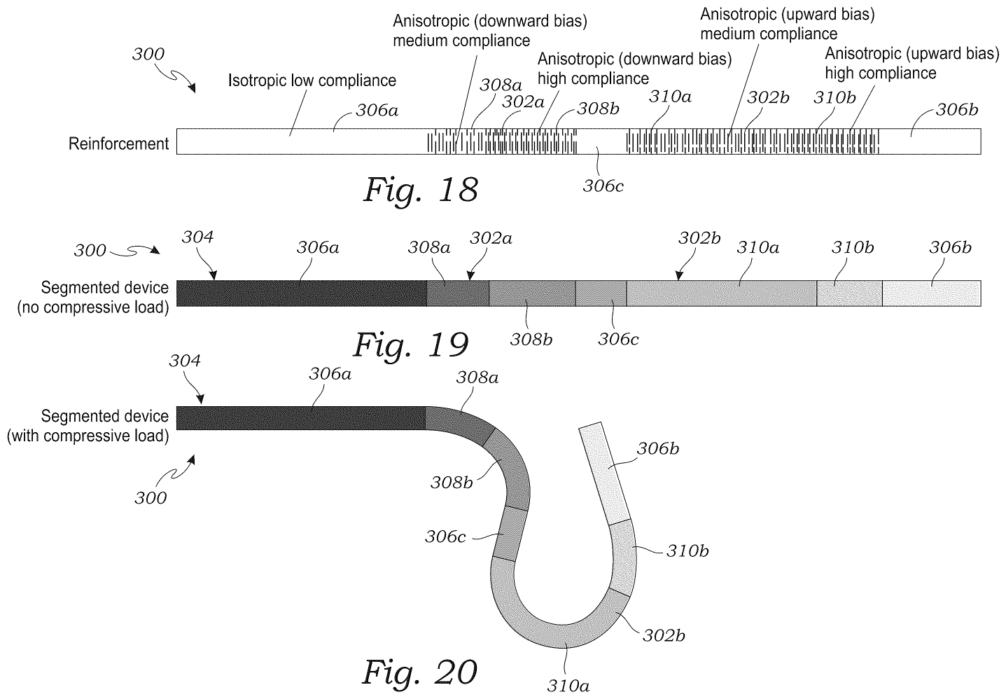

Referring to FIG. 18, a mechanical actuation system 300 for providing an anchoring feature for a catheter system is illustrated. The catheter system may be similar to the catheter system 100, described herein, except that the actuators 114 are replaced by the mechanical actuation system 300. The mechanical actuation system 300 includes a tube 304 having one or more mechanical actuator elements 302. The tube 304 may be a hypotube or other suitable tube for use in a catheter. The illustrated embodiment includes two actuators elements 302a and 302b. The mechanical actuation system 300 may include any suitable number of mechanical actuators 302 to provide the desired anchoring shape for anchoring in a body lumen having corresponding shape.

In the illustrated embodiment, the actuators elements 302 are integrally formed in the tube 304 such tube has separate longitudinal regions, including regions comprising the actuator elements 302 and non-actuated regions 306a, 306b, 306c, located adjacent to the actuator elements 302. The non-actuated regions 306a, 306b, 306c are regions of the tube 304 where the axial compressive compliance is low and isotropic about the center axis of the tube 304, which form straight non-actuating segments of the tube 304.

The actuator elements 302 are regions of the tube 304 where the compliance is anisotropic about the center axis and may include areas of varying compliance. The actuator element 302a has a first anisotropic area 308a having a downward bias and a medium compliance (i.e., bends more for a given compressive force than the low compliance, and less than the high compliance), and a second anisotropic area 308b having a downward bias and a high compliance (i.e., bends more for given compressive force than the medium compliance). The direction of the “bias” is based on the orientation as shown in FIGS. 18-20. In other words, the first anisotropic area 308a and second anisotropic area 308b are configured to bend clockwise in response to a compressive force, as shown in FIG. 20. As illustrated in FIG. 20, for a given compressive force, the first anisotropic area 308a will bend less (i.e., bend at a larger bending radius or bending curvature) than the second anisotropic area 308b. Similarly, the actuator element 302b has a first anisotropic area 310a having an upward bias and a medium compliance (i.e., bends more for a given compressive force than the low compliance, and less than the high compliance), and a second anisotropic area 310b having an upward bias and a high compliance (i.e., bends more for given compressive force than the medium compliance). In the orientation as shown in FIGS. 16-18, the first anisotropic area 310a and second anisotropic area 3108b are configured to bend counter-clockwise in response to a compressive force, as shown in FIG. 18. As depicted in FIG. 18, for a given compressive force, the first anisotropic area 310a will bend less (i.e., bend at a larger bending radius or bending curvature) than the second anisotropic area 310b.

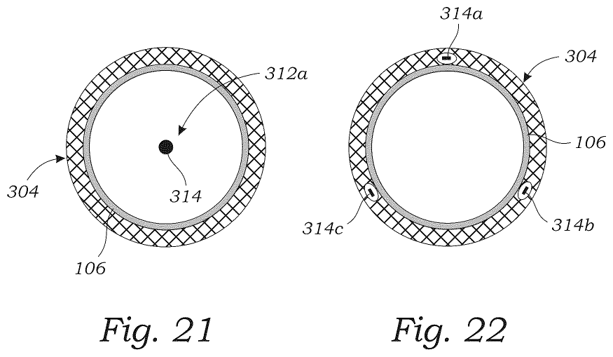

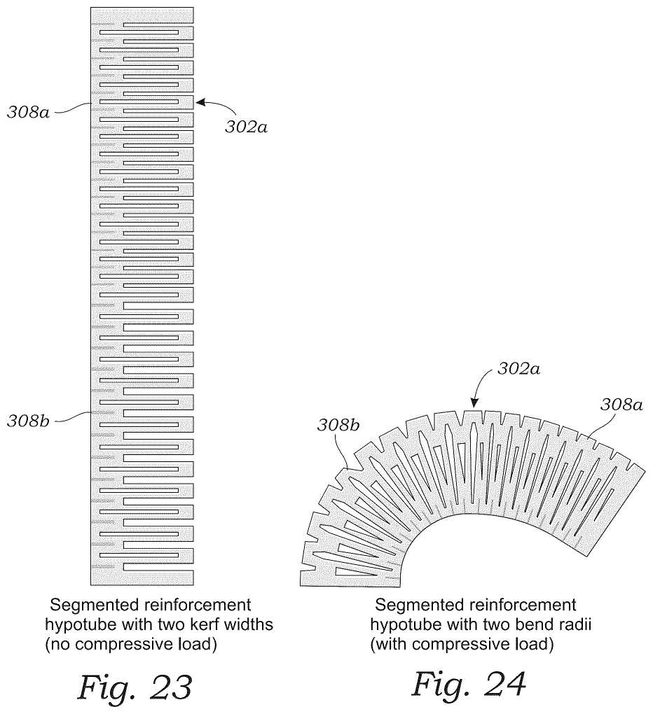

FIG. 19 shows the relative axial compressive compliance of each region of the mechanical actuation system 300. As shown in FIG. 20, upon actuating the actuation system 300 by applying an axial compressive force on the tube 303, the actuator elements 302 will bend based on the configuration of each respective actuator element 302. Turning to FIGS. 19 and 20, two alternative actuation mechanisms 312a, 312b are illustrated. The actuation mechanism 312a of FIG. 21 includes a single pull wire 314 disposed within the lumen of the catheter. The pull wire 314 has a distal end attached to the distal end of the actuation system 300 and extends proximally along the length of the catheter to the proximal end of the catheter where it can be operated to apply and remove a compressive force to the actuation system 300. The actuation mechanism 312b of FIG. 22 has a plurality of pull wires 314a, 314b, 314c disposed within passages or lumens in the wall of the tube 304. The pull wires 314a, 314b and 314c are attached and operated similar to the pull wire 314 of FIG. 19. The plurality of pull wires 14a, 314b, 314c may all be actuated simultaneously or each sequentially and similarly the actuation forces on the individual wires may all have the same or each may have different magnitudes.

Turning to FIGS. 24-25, an exemplary embodiment of an actuator element 302a is illustrated. The anisotropic areas 308a, 308b of the actuator element 302a are formed by a plurality of cuts in the tube 304, such as laser or machine cuts in a hypotube 304. The width, spacing and location of the cuts establish the varying compressive compliance and bending curvature of each anisotropic area 308. As shown in FIG. 22, upon application of a compressive force, the anisotropic area 308a bends with larger radius than the anisotropic area 308b.

The anisotropic areas 308 can impart bending bias in a given direction for each area of the actuator element 302, which can vary from area to area to cause bends in different planes at each area or vary continuously within a bending region to create complex bending, including forming a helix shape along a portion or all of the actuation system 300.

A method of using a catheter system 100 having the actuation system 300 is similar to using the catheters 120 or 140 described above, except that the actuation system 300 is actuated by actuating the pull wire(s) 314 to apply and remove an axial compressive force on the actuator elements 302. The distal end 110 of the catheter 102 is inserted into the body lumen with no compressive load applied to the actuation system 300 such that the actuation elements 302 are non-actuated and the anchoring portion 120 of the tubular member 106 has its relaxed shape and/or intrinsic flexibility. The catheter 102 is advanced into the body lumen to position the catheter 102 in a desired location with the anchoring portion positioned in the body lumen 122. The actuation system 300 is then actuated by applying a compressive force to the actuation elements 302, thereby causing the actuation elements 302 to bend and stiffen into an anchoring shape which anchors the anchoring portion 120 in place in the body lumen 122.

Similarly, another method of using a catheter system 100 is directed to a catheter system having an actuation system 300 is configured to form the catheter 102 into a helical shape such that the tubular member 106 can make circumferential contact with a wall of a body lumen along a length of tubular member 106 sufficient to stabilize the tubular member in a body lumen. The method includes a step of inserting the catheter 102 into a body lumen 122 with the actuation system 300 non-actuated such that the tubular member 106 has its relaxed shape and/or intrinsic flexibility. The catheter 100 is advanced into the body lumen to a desired location within the body lumen. The actuation system 300 is then actuated by applying a compressive force to the actuation elements 302, thereby causing the actuation elements 302 to bend and stiffen into the helical shape such that the catheter 100 makes circumferential contact with the wall of the body lumen]. The circumferential contact stabilizes and/or anchors the catheter 100 in the body lumen.

Although particular embodiments have been shown and described, it is to be understood that the above description is not intended to limit the scope of these embodiments. While embodiments and variations of the many aspects of the invention have been disclosed and described herein, such disclosure is provided for purposes of explanation and illustration only. Thus, various changes and modifications may be made without departing from the scope of the claims. For example, not all of the components described in the embodiments are necessary, and the invention may include any suitable combinations of the described components, and the general shapes and relative sizes of the components of the invention may be modified. Accordingly, embodiments are intended to exemplify alternatives, modifications, and equivalents that may fall within the scope of the claims. The invention, therefore, should not be limited, except to the following claims, and their equivalents.

What is claimed is:

1. A catheter system, comprising:
an elongated, flexible tubular member, having an anchoring portion with an intrinsic flexibility;
one or more actuators disposed on the tubular member, the one or more actuators having a non-actuated state in which the one or more actuators allow the tubular member to have its relaxed shape, and an actuated state in which, in response to being actuated, the one or more actuators modify a shape of the anchoring portion into an anchoring shape and also stiffen the anchoring portion into the anchoring shape, the anchoring shape configured to anchor the anchoring portion in place in a body lumen corresponding to the anchoring shape,
wherein the catheter system has (a) a non-actuated configuration wherein the one or more actuators are non-actuated such that the anchoring portion of the tubular member has its intrinsic flexibility, and (b) an actuated configuration wherein the one or more actuators are actuated thereby causing the actuators to stiffen the anchoring portion in the anchoring shape configured to anchor the anchoring portion in place in a body lumen corresponding to the anchoring shape.

2. The catheter system of claim 1, wherein the one or more actuators comprise electroactive polymer actuators which are actuated by one or more electrical control signals.

3. The catheter system of claim 2, further comprising:
a controller operably coupled to the plurality of electroactive polymer actuators, the controller configured to selectively transmit the electrical control signals to the plurality of electroactive polymer actuators.

4. The catheter system of claim 3, wherein the controller is coupled to the one or more electroactive polymer actuators by a plurality of conductors, each conductor having a first end connected to respective electroactive polymer actuator and a second end connected to the controller.

5. The catheter system of claim 4, wherein the electrical control signals comprise a respective voltage applied to the one or electroactive polymer actuators.

6. The catheter system of claim 1, wherein the one or more actuators comprise pressure actuators which are actuated by one or more pressure lumens coupled to the pressure actuators.

7. A catheter system, comprising:
an elongated, flexible tubular member, having an anchoring portion with an intrinsic flexibility;
one or more mechanical actuators disposed on the tubular member, the mechanical actuators actuated by one or more pull wires coupled to the mechanical actuators, the one or more mechanical actuators having a non-actuated state in which the one or more actuators allow the tubular member to have its relaxed shape, and an actuated state in which, in response to being actuated, the one or more mechanical actuators stiffen the anchoring portion into an anchoring shape configured to anchor the anchoring portion in place in a body lumen corresponding to the anchoring shape,
wherein the catheter system has (a) a non-actuated configuration wherein the one or more actuators are non-actuated such that the anchoring portion of the tubular member has its intrinsic flexibility, and (b) an actuated configuration wherein the one or more actuators are actuated thereby causing the actuators to stiffen the anchoring portion in the anchoring shape configured to anchor the anchoring portion in place in a body lumen corresponding to the anchoring shape.

8. The catheter system of claim 7, wherein each mechanical actuator comprises an actuator element having an anisotropic region about a center axis of the tubular member with a first area having a first compliance and a second area having a second compliance different from the first compliance such that a compressive loading on the actuator element by the one or more pull wires causes the actuator element to bend and stiffen into a predetermined shape.

9. The catheter system of claim 8, wherein each actuator element comprises a region of a hypotube having cut patterns which confer a first compliance to the first area and a second compliance to the second area.

10. The catheter system of claim 7, wherein the one or more mechanical actuators are configured to also modify a shape of the anchoring portion, and wherein in the actuated configuration the one or more mechanical actuators also form the anchoring portion into the anchoring shape.

11. A catheter system, comprising:
an elongated, flexible tubular member;
a plurality of electroactive polymer actuators disposed on the tubular member, the plurality of electroactive polymer actuators configured to form the tubular member into a substantially sinusoidal shape and to transition a shape of the tubular member back and forth between a substantially sinusoidal shape 0° out of phase and a substantially sinusoidal shape 180° out of phase;
a controller operably coupled to the plurality of electroactive polymer actuators, the controller configured to transmit control signals to the plurality of electroactive polymer actuators to dynamically cycle the tubular member back and forth between the substantially sinusoidal shape 0° out of phase and the substantially sinusoidal shape 180° out of phase.

12. The catheter system of claim 11, wherein the controller dynamically cycles the sinusoidal shape of the tubular member at an amplitude and frequency which reduces static friction of the tubular member as it is moved longitudinally within a body lumen.

13. The catheter system of claim 12, wherein the plurality of electroactive polymer actuators comprise a first set and a second set, and on a first side of the tubular member electroactive polymer actuators of the first set are disposed longitudinally along the tubular member and alternating with electroactive polymer actuators of the second set disposed longitudinally along the tubular member on the first side, and on a second side of the tubular member opposite the first side, electroactive polymer actuators of the first set are disposed longitudinally along the tubular member and alternating with electroactive polymer actuators of the second set disposed along the tubular member on the second side, such that actuation of the first set of electroactive polymer actuators modifies the shape of the tubular member into the substantially sinusoidal shape 0° out of phase, and actuation of the second set of electroactive polymer actuators modifies the shape of the tubular member into the sinusoidal shape 180 out of phase.

14. The catheter system of claim 13, wherein the first set of electroactive polymer actuators are electrically connected to the controller via a first pair of electrical connections such that all of the electroactive polymer actuators in the first set are actuated by a single first signal from the controller, and the second set of electroactive polymer are connected to the controller via a second pair of electrical connections such that all of the electroactive polymer actuators in the first set are actuated by a single second signal from the controller, and wherein the second pair of electrical connections are electrically insulated/separated from the first pair of electrical connections.

15. A catheter system, comprising:
an elongated, flexible tubular member;
an actuator disposed on the tubular member in a helical path along the tubular member such that actuation of the actuator transitions a shape of the tubular member into a substantially helical shape such that the tubular member can make circumferential contact with a wall of a body lumen along a length of tubular member sufficient to stabilize the tubular member in a body lumen.

16. The catheter system of claim 15, wherein the catheter is configured such that the tubular member in the substantially helical shape makes circumferential contact with a wall of a body lumen along substantially an entire insertable length of the tubular member.

17. The catheter system of claim 15, wherein the actuator comprises an electroactive polymer actuator which is actuated by one or more electrical control signals.

18. The catheter system of claim 17, further comprising:
a controller operably coupled to the electroactive polymer actuator, the controller configured to selectively transmit an electrical control signal to the electroactive polymer actuator.

19. The catheter system of claim 18, wherein the controller is coupled to the electroactive polymer actuator by a pair of conductors, each conductor having a first end connected to the electroactive polymer actuator and a second end connected to the controller.

20. The catheter system of claim 18, wherein the electrical control signal comprises a respective voltage applied to the electroactive polymer actuator.
